SEQUENCE LISTING (5 sequences)

SEQ ID NO: 1 - protein, 7 aa, Artificial
  Synthetic
RTDLXXL

SEQ ID NO: 2 - protein, 4 aa, Artificial
  Synthetic
RXDL

SEQ ID NO: 3 - protein, 7 aa, Artificial
  Synthetic
RXDLXXX

SEQ ID NO: 4 - protein, 10 aa, Artificial
  Synthetic
WREPSFAMLS

SEQ ID NO: 5 - protein, 10 aa, Artificial
  Synthetic
WREPGRMELN

(19) United States
(12) Patent Application Publication
(10) Pub. No.: US 2006/0140909 A1
(43) Pub. Date: Jun. 29, 2006

(54) Method of using adenoviral vectors with increased persistence in vivo

(75) Inventors: Thomas J. Wickham, Billerica, MA (US); Masaki Akiyama, Gaithersburg, MD (US); Jason G.D. Gall, Germantown, MD (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignees: GenVec, Inc., Gaithersburg, MD (US); FUSO Pharmaceutical Industries, Ltd., Osaka (JP)

(21) Appl. No.: 11/208,405

(22) Filed: Aug. 19, 2005

Related U.S. Application Data
(63) Continuation of application No. PCT/US04/04922, filed on Feb. 18, 2004.
Application No. PCT/US04/04922 is a continuation of application No. 10/374,271, filed on Feb. 25, 2003.

(51) Int. Cl. A61K 48/00; C12N 15/861

(52) U.S. Cl. 424/93.2; 435/456

(57) ABSTRACT

The invention provides a method of expressing an exogenous nucleic acid in a mammal. The method comprises slowly releasing into the bloodstream a dose of replication-deficient or conditionally-replicating adenoviral vector having reduced ability to transduce mesothelial cells and hepatocytes. The normalized average bloodstream concentration of the adenovirus over 24 hours post-administration is at least about 1%. Alternatively, the normalized average bloodstream concentration over 24 hours post-administration is at least about 5-fold greater than the normalized average bloodstream concentration for an equivalent dose of a wild-type adenoviral vector. A method of destroying tumor cells in a mammal also is provided, as is a replication-deficient adenoviral vector comprising a serotype 5 or serotype 35 adenoviral genome with a serotype 41 fiber protein, wherein the replication-deficient adenoviral vector exhibits reduced native binding to integrins.

### CROSS-REFERENCE TO RELATED APPLICATIONS

This patent application is a continuation of International Patent Application No. PCT/US2004/004922, filed Feb. 18, 2004, designating the U.S., which claims the benefit of copending U.S. patent application Ser. No. 10/374,271, filed Feb. 25, 2003.

### FIELD OF THE INVENTION

This invention pertains to methods of achieving increased persistence of adenoviral vectors in circulation.

### BACKGROUND OF THE INVENTION

Gene therapy is gaining acceptance in the scientific community as a promising treatment for a variety of ailments. Gene transfer vectors derived from adenovirus have proven to have many attractive characteristics in the context of gene therapy including substantial and transient gene expression, the ability to be propagated in high titers, and the ability to transduce a wide variety of cell types. Despite these advantageous characteristics, adenoviral vectors suffer from limitations similar to those of other gene transfer vectors with respect to achieving widespread delivery in the body.

Viral vectors inherently encode and/or display antigenic epitopes that are recognized by a host immune system. The immunogenicity of viral vectors, including adenoviral vectors, is a major impediment in the use of these gene transfer vehicles in vivo. For example, a majority of the human population has been exposed to adenovirus and, therefore, has pre-existing immunity to adenoviral vectors based on human adenovirus serotypes, which limits the effectiveness of the virus as a gene transfer vector. Aside from pre-existing immunity, adenoviral vector administration induces inflammation and activates both innate and acquired immune mechanisms. Adenoviral vectors activate antigen-specific (e.g., T-cell dependent) immune responses, which limit the duration of transgene expression following an initial administration of the vector. In addition, exposure to adenoviral vectors stimulates production of neutralizing antibodies by B cells, which precludes gene expression from subsequent doses of adenoviral vector (Wilson & Kay, Nat. Med., 3(9), 887-889 (1995)). Indeed, the effectiveness of repeated administration of the vector can be severely limited by host immunity. For example, animal studies demonstrate that intravenous or local administration of an adenoviral serotype 2 or 5 vector can result in the production of neutralizing antibodies directed against the vector which prevent expression from the same serotype vector administered 1 to 2 weeks later (see, for example, Kass-Eisler et al., Gene Therapy, 1, 395-402 (1994), and Kass-Eisler et al., Gene Therapy, 3, 154-162 (1996)).

In addition to stimulation of humoral immunity, cell-mediated immune functions are responsible for clearance of the virus from the body. Rapid clearance of the virus is attributed to innate immune mechanisms (see, e.g., Worgall et al., Human Gene Therapy, 8, 37-44 (1997)), and likely involves Kupffer cells found within the liver. Adenoviral vectors are typically cleared from circulation within minutes and are cleared from the body within about 7-10 days. Within the first two days of infection, approximately 90% of adenoviral vector DNA is eliminated (Elkon et al., PNAS, 94, 9814-9819 (1997)). The rapid clearance of adenoviral vectors decreases circulation time and prevents efficient delivery to target cells via systemic circulation, which may be required to treat diseases such as disseminated cancers.

To address the shortcomings of adenoviral vectors with respect to persistence in the body, modification of the antigenic determinants of adenoviral particles has been proposed. It is reasoned that avoidance of clearance mechanisms of the body will increase the amount of time in circulation, thereby increasing the likelihood of transducing target cells distal to the point of administration. Adenoviral fiber, penton, and hexon proteins have received the most attention as these represent the first exposure of the virus to the host's immune and clearance systems. For example, U.S. Pat. No. 6,153,435 (Crystal et al.) describes adenoviral vectors having a chimeric adenovirus coat protein with a decreased ability or inability to be recognized by a neutralizing antibody directed against the corresponding wild-type adenovirus coat protein. Genetic manipulation of adenoviral coat proteins has resulted in success, although somewhat limited, in avoiding host immunity.

Despite advances in modulating the antigenicity of adenoviral vectors, an improved method of using adenoviral vectors in vivo is required to increase retention of adenoviral vectors in the body, obtain better distribution, and increase target cell transduction. The invention provides such a method of using adenoviral vectors to obtain increased persistence in circulation. These and other advantages of the invention, as well as additional inventive features, will be apparent from the description of the invention provided herein.

### BRIEF SUMMARY OF THE INVENTION

The invention provides a method of expressing an exogenous nucleic acid in a mammal. The method comprises slowly releasing into the bloodstream of the mammal a dose of replication-deficient or conditionally-replicating adenoviral vector. The adenoviral vector has a reduced ability to transduce mesothelial cells and hepatocytes compared to wild-type adenovirus. The replication-deficient or conditionally-replicating adenoviral vector further comprises an exogenous nucleic acid. The normalized average bloodstream concentration of the replication-deficient or conditionally-replicating adenovirus over a time period of 24 hours post-administration is at least 1%. Alternatively, the normalized average bloodstream concentration of the replication-deficient or conditionally-replicating adenovirus over a time period of 24 hours post-administration is at least about 5-fold greater than the normalized average bloodstream concentration for an equivalent dose of a wild-type adenovirus. A host cell in the mammal is transduced by the replication-deficient or conditionally-replicating adenoviral vector, and the exogenous nucleic acid is expressed.

The invention further provides a method of destroying tumor cells in a mammal. The method comprises slowly delivering a dose of a replication-deficient or conditionally-replicating adenoviral vector to the bloodstream comprising (a) a nucleic acid sequence encoding a tumoricidal agent and (b) an adenoviral fiber protein which does not mediate adenoviral entry via a coxsackievirus and adenovirus receptor (CAR), such that the tumoricidal agent is produced and tumor cells in the mammal are destroyed.

The invention also provides a replication-deficient adenoviral vector comprising a serotype 5 or serotype 35 adenoviral genome with a serotype 41 fiber protein, wherein the replication-deficient adenoviral vector exhibits reduced native binding to integrins.

### BRIEF DESCRIPTION OF THE DRAWINGS

FIG. 1 is a graph of percent (%) injected dose of AdL and AdL.F*PB* versus minutes following intravenous injection of the adenoviral vectors.

FIG. 2 is a graph of percent (%) injected dose of AdL, AdL.F*, and AdL.F*PB* versus minutes following intraperitoneal injection of the adenoviral vectors.

FIG. 3 is a graph of percent (%) injected dose of AdL, AdL.F*, and AdL.F*PB* versus minutes following intraperitoneal injection of the adenoviral vectors. Ten minutes prior to administration of the adenoviral vectors, a pre-dose of null adenoviral vector was administered.

FIG. 4 is a graph of percent (%) injected dose of 1×10¹⁰ particle units (pu) or 1×10¹¹ pu of AdL or AdL.F*PB*, with or without a pre-dose of null adenoviral vector (Null), versus minutes post-vector injection.

FIG. 5 is a bar graph illustrating relative light units (RLU)/mg of protein in samples taken from tumor, liver, spleen, kidney, and lung tissue and generated by intraperitoneal delivery of AdL, AdL.F*PB*, AdL.**RGD, or AdL.**αvβ6.

FIG. 6 is a bar graph illustrating relative light units (RLU)/mg of protein in samples taken from tumor, liver, spleen, kidney, and lung tissue and generated by intravenous delivery of AdL, AdL.F*PB*, AdL.**RGD, or AdL.**αvβ6.

### DETAILED DESCRIPTION OF THE INVENTION

The invention is predicated, at least in part, on the surprising discovery that gene transfer vectors, in particular adenoviral gene transfer vectors, can be delivered to systemic circulation of a mammal such that a greater fraction of a dose of gene transfer vector remains in the bloodstream for at least 24 hours post-administration than previously achieved. Adenoviral vectors are typically cleared from circulation within minutes. The inability to retain adenoviral vectors in circulation limits the effectiveness of a dose of an adenoviral gene transfer vector in delivering a transgene to target cells, particularly target cells distal to the point of administration. For example, the most effective means of delivering a dose of adenoviral vector to a target tissue was directly injecting the virus into the tissue such that a majority of the dose contacts the target cells. However, when target tissue is not readily accessible for injection, or in instances wherein target cells are scattered throughout the body, injection directly into target tissue is not feasible. The invention provides a method of delivering an adenoviral gene transfer vector to the circulatory system of a mammal for distribution throughout the body, but which allows maximal retention of the dose of adenoviral vector to increase the likelihood of target cell transduction. Adenoviral vectors that remain in circulation for several minutes, preferably several hours or more, i.e., 1, 3, 5, or 7 days, post-administration and remain able to transduce cells or propagate are said to have a prolonged half-life in vivo, increased persistence, or an extended circulation time.

In particular, the invention provides a method of expressing an exogenous nucleic acid in a mammal. The method comprises slowly releasing into the bloodstream of the mammal a dose of replication-deficient or conditionally-replicating adenoviral vector comprising an exogenous nucleic acid. The replication-deficient or conditionally-replicating adenoviral vector has a reduced ability to transduce mesothelial cells and hepatocytes compared to wild-type adenovirus. The normalized average bloodstream concentration of the replication-deficient or conditionally-replicating adenovirus over a time period of 24 hours post-administration is at least 1%. A host cell in the mammal is transduced and the exogenous nucleic acid is expressed therein.

Adenoviral Vector

Adenovirus from any origin, any subtype, mixture of subtypes, or any chimeric adenovirus can be used as the source of the viral genome for the replication-deficient or conditionally-replicating adenoviral vector. While non-human adenovirus (e.g., simian, avian, canine, ovine, or bovine adenoviruses) can be used to generate the replication-deficient adenoviral vector, a human adenovirus preferably is used as the source of the viral genome for the replication-deficient or conditionally-replicating adenoviral vector of the inventive method. The adenovirus can be of any subgroup or serotype. For instance, an adenovirus can be of subgroup A (e.g., serotypes 12, 18, and 31), subgroup B (e.g., serotypes 3, 7, 11, 14, 16, 21, 34, 35, and 50), subgroup C (e.g., serotypes 1, 2, 5, and 6), subgroup D (e.g., serotypes 8, 9, 10, 13, 15, 17, 19, 20, 22-30, 32, 33, 36-39, and 42-48), subgroup E (e.g., serotype 4), subgroup F (e.g., serotypes 40 and 41), an unclassified serogroup (e.g., serotypes 49 and 51), or any other adenoviral serotype. Adenoviral serotypes 1 through 51 are available from the American Type Culture Collection (ATCC, Manassas, Va.). Preferably, the adenoviral vector is of human subgroup C, especially serotype 2 or even more desirably serotype 5. Adenoviral vectors of serotype 35 or serotype 41 also is appropriate for use in the context of the invention.

By “replication-deficient” is meant that the adenoviral vector comprises an adenoviral genome that lacks at least one replication-essential gene function (i.e., such that the adenoviral vector does not replicate in typical host cells, especially those in a human patient that could be infected by the adenoviral vector in the course of treatment in accordance with the invention). A deficiency in a gene, gene function, or gene or genomic region, as used herein, is defined as a deletion of sufficient genetic material of the viral genome to impair or obliterate the function of the gene whose nucleic acid sequence was deleted in whole or in part. Replication-essential gene functions are those gene functions that are required for replication (e.g., propagation) and are encoded by, for example, the adenoviral early regions (e.g., the E1, E2, and E4 regions), late regions (e.g., the L1-L5 regions), genes involved in viral packaging (e.g., the IVa2 gene), and virus-associated RNAs (e.g., VA-RNA1 and/or VA-RNA2). More preferably, the replication-deficient adenoviral vector comprises an adenoviral genome deficient in at least one replication-essential gene function of one or more regions of the adenoviral genome. Preferably, the adenoviral vector is deficient in at least one gene function of the E1 region of the adenoviral genome required for viral replication (denoted an E1-deficient adenoviral vector). In addition to such a deficiency in the E1 region, the recombinant adenovirus also can have a mutation in the major late promoter (MLP), as discussed in International Patent Application WO 00/00628. Most preferably, the adenoviral vector is deficient in at least one replication-essential gene function (desirably all replication-essential gene functions) of the E1 region and at least part of the nonessential E3 region (e.g., an Xba I deletion of the E3 region) (denoted an E1/E3-deficient adenoviral vector). With respect to the E1 region, the adenoviral vector can be deficient in part or all of the E1A region and part or all of the E1B region, e.g., in at least one replication-essential gene function of each of the E1A and E1B regions. When the adenoviral vector is deficient in at least one replication-essential gene function in one region of the adenoviral genome (e.g., an E1- or E1/E3-deficient adenoviral vector), the adenoviral vector is referred to as “singly replication-deficient.” A particularly preferred singly replication-deficient adenoviral vector is that described in the Examples herein.

The adenoviral vector can be “multiply replication-deficient,” meaning that the adenoviral vector is deficient in one or more replication-essential gene functions in each of two or more regions of the adenoviral genome. For example, the aforementioned E1-deficient or E1/E3-deficient adenoviral vector can be further deficient in at least one replication-essential gene function of the E4 region (denoted an E1/E4- or E1/E3/E4-deficient adenoviral vector), and/or the E2 region (denoted an E1/E2- or E1/E2/E3-deficient adenoviral vector), preferably the E2A region (denoted an E1/E2A- or E1/E2A/E3-deficient adenoviral vector). Ideally, the adenoviral vector lacks replication-essential gene functions of only those replication-essential gene functions encoded by the early regions of the adenoviral genome, although this is not required in all contexts of the invention. A preferred multiply-deficient adenoviral vector comprises an adenoviral genome having deletions of nucleotides 457-3332 of the E1 region, nucleotides 28593-30470 of the E3 region, nucleotides 32826-35561 of the E4 region, and, optionally, nucleotides 10594-10595 of the region encoding VA-RNA1. However, other deletions may be appropriate. Nucleotides 356-3329 or 356-3510 can be removed to create a deficiency in replication-essential E1 gene functions. Nucleotides 28594-30469 can be deleted from the E3 region of the adenoviral genome. While the specific nucleotide designations recited above correspond to the adenoviral serotype 5 genome, the corresponding nucleotides for non-serotype 5 adenoviral genomes can easily be determined by those of ordinary skill in the art.

The adenoviral vector, when multiply replication-deficient, especially in replication-essential gene functions of the E1 and E4 regions, preferably includes a spacer element to provide viral growth in a complementing cell line similar to that achieved by singly replication-deficient adenoviral vectors, particularly an E1-deficient adenoviral vector. The spacer element can contain any sequence or sequences which are of a desired length, such as sequences at least about 15 base pairs (e.g., between about 15 base pairs and about 12,000 base pairs), preferably about 100 base pairs to about 10,000 base pairs, more preferably about 500 base pairs to about 8,000 base pairs, even more preferably about 1,500 base pairs to about 6,000 base pairs, and most preferably about 2,000 to about 3,000 base pairs in length. The spacer element sequence can be coding or non-coding and native or non-native with respect to the adenoviral genome, but does not restore the replication-essential function to the deficient region. In the absence of a spacer, production of fiber protein and/or viral growth of the multiply replication-deficient adenoviral vector is reduced by comparison to that of a singly replication-deficient adenoviral vector. However, inclusion of the spacer in at least one of the deficient adenoviral regions, preferably the E4 region, can counteract this decrease in fiber protein production and viral growth. The use of a spacer in an adenoviral vector is described in, e.g., U.S. Pat. No. 5,851,806. In one embodiment of the inventive method, the replication-deficient or conditionally-replicating adenoviral vector is an E1/E4-deficient adenoviral vector wherein the L5 fiber region is retained, and a spacer is located between the L5 fiber region and the right-side ITR. More preferably, in such an adenoviral vector, the E4 polyadenylation sequence alone or, most preferably, in combination with another sequence, exists between the L5 fiber region and the right-side ITR, so as to sufficiently separate the retained L5 fiber region from the right-side ITR, such that viral production of such a vector approaches that of a singly replication-deficient adenoviral vector, particularly an E1-deficient adenoviral vector.

The adenoviral vector can be deficient in replication-essential gene functions of only the early regions of the adenoviral genome, only the late regions of the adenoviral genome, and both the early and late regions of the adenoviral genome. The adenoviral vector also can have essentially the entire adenoviral genome removed, in which case it is preferred that at least either the viral inverted terminal repeats (ITRs) and one or more promoters or the viral ITRs and a packaging signal are left intact (i.e., an adenoviral amplicon). The 5′ or 3′ regions of the adenoviral genome comprising ITRs and packaging sequence need not originate from the same adenoviral serotype as the remainder of the viral genome. For example, the 5′ region of an adenoviral serotype 5 genome (i.e., the region of the genome 5′ to the adenoviral E1 region) can be replaced with the corresponding region of an adenoviral serotype 2 genome (e.g., the Ad5 genome region 5′ to the E1 region of the adenoviral genome is replaced with nucleotides 1-456 of the Ad2 genome). Suitable replication-deficient adenoviral vectors, including multiply replication-deficient adenoviral vectors, are disclosed in U.S. Pat. Nos. 5,837,511; 5,851,806; 5,994,106; and 6,127,175; U.S. Published Patent Applications 2001/0043922 A1; 2002/0004040 A1; 2002/0031831 A1; and 2002/0110545 A1, and International Patent Applications WO 94/28152; WO 95/02697; WO 95/34671; WO 96/22378; WO 97/12986; and WO 97/21826. Ideally, the replication-deficient or conditionally-replicating adenoviral vector is used in the context of the invention in the form of an adenoviral vector composition, especially a pharmaceutical composition, which is virtually free of replication-competent adenovirus (RCA) contamination (e.g., the composition comprises less than about 1% of RCA contamination). Most desirably, the composition is RCA-free. Adenoviral vector compositions and stocks that are RCA-free are described in U.S. Pat. Nos. 5,944,106 and 6,482,616, U.S. Published Patent Application 2002/0110545 A1, and International Patent Application WO 95/34671.

Replication-deficient adenoviral vectors are typically produced in complementing cell lines that provide gene functions not present in the replication-deficient adenoviral vectors, but required for viral propagation, at appropriate levels in order to generate high titers of viral vector stock. A preferred cell line complements for at least one and preferably all replication-essential gene functions not present in a replication-deficient adenovirus. The complementing cell line can complement for a deficiency in at least one replication-essential gene function encoded by the early regions, late regions, viral packaging regions, virus-associated RNA regions, or combinations thereof, including all adenoviral functions (e.g., to enable propagation of adenoviral amplicons). Most preferably, the complementing cell line complements for a deficiency in at least one replication-essential gene function (e.g., two or more replication-essential gene functions) of the E1 region of the adenoviral genome, particularly a deficiency in a replication-essential gene function of each of the E1A and E1 B regions. In addition, the complementing cell line can complement for a deficiency in at least one replication-essential gene function of the E2 (particularly as concerns the adenoviral DNA polymerase and terminal protein) and/or E4 regions of the adenoviral genome. Desirably, a cell that complements for a deficiency in the E4 region comprises the E4-ORF6 gene sequence and produces the E4-ORF6 protein. Such a cell desirably comprises at least ORF6 and no other ORF of the E4 region of the adenoviral genome. The cell line preferably is further characterized in that it contains the complementing genes in a non-overlapping fashion with the adenoviral vector, which minimizes, and practically eliminates, the possibility of the vector genome recombining with the cellular DNA. Accordingly, the presence of replication competent adenoviruses (RCA) is minimized if not avoided in the vector stock, which, therefore, is suitable for certain therapeutic purposes, especially gene therapy purposes. The lack of RCA in the vector stock avoids the replication of the adenoviral vector in non-complementing cells. Construction of such a complementing cell lines involve standard molecular biology and cell culture techniques, such as those described by Sambrook et al., Molecular Cloning, a Laboratory Manual, 2d edition, Cold Spring Harbor Press, Cold Spring Harbor, N.Y. (1989), and Ausubel et al., Current Protocols in Molecular Biology, Greene Publishing Associates and John Wiley & Sons, New York, N.Y. (1994).

Complementing cell lines for producing the adenoviral vector include, but are not limited to, 293 cells (described in, e.g., Graham et al., J. Gen. Virol., 36, 59-72 (1977)), PER.C6 cells (described in, e.g., International Patent Application WO 97/00326, and U.S. Pat. Nos. 5,994,128 and 6,033,908), and 293-ORF6 cells (described in, e.g., International Patent Application WO 95/34671 and Brough et al., J. Virol., 71, 9206-9213 (1997)). In some instances, the complementing cell will not complement for all required adenoviral gene functions. Helper viruses can be employed to provide the gene functions in trans that are not encoded by the cellular or adenoviral genomes to enable replication of the adenoviral vector. Adenoviral vectors can be constructed, propagated, and/or purified using the materials and methods set forth, for example, in U.S. Pat. Nos. 5,965,358, 5,994,128, 6,033,908, 6,168,941, 6,329,200, 6,383,795, 6,440,728, 6,447,995, and 6,475,757, U.S. Patent Application Publication No. 2002/0034735 A1, and International Patent Applications WO 98/53087, WO 98/56937, WO 99/15686, WO 99/54441, WO 00/12765, WO 01/77304, and WO 02/29388, as well as the other references identified herein. Non-group C adenoviral vectors, including adenoviral serotype 35 vectors, can be produced using the methods set forth in, for example, U.S. Pat. Nos. 5,837,511 and 5,849,561, and International Patent Applications WO 97/12986 and WO 98/53087. Moreover, numerous adenoviral vectors are available commercially.

If the adenoviral vector is not replication-deficient, ideally the adenoviral vector is manipulated to limit replication of the vector to within the target tissue. For example, the adenoviral vector can be a conditionally-replicating adenoviral vector, which is engineered to replicate under conditions pre-determined by the practitioner. For example, replication-essential gene functions, e.g., gene functions encoded by the adenoviral early regions, can be operably linked to an inducible, repressible, or tissue-specific transcription control sequence, e.g., promoter. In this embodiment, replication requires the presence or absence of specific factors that interact with the transcription control sequence. Conditionally-replicating adenoviral vectors are particularly useful in delivering exogenous nucleic acids with the purpose of destroying target cells. Replication of the adenoviral vector can be limited to a target tissue, thereby allowing greater distribution of the vector throughout the tissue while exploiting adenovirus' natural ability to lyse cells during the replication cycle. In cancer therapy, conditionally-replicating adenovirus provides a mode of destroying tumor cells in addition to delivery of lethal exogenous nucleic acids. Conditionally-replicating adenoviral vectors are described further in U.S. Pat. No. 5,998,205.

The replication-deficient or conditionally-replicating adenoviral vector has a reduced ability to transduce mesothelial cells and hepatocytes compared to wild-type adenovirus of the same serotype of the replication-deficient or conditionally-replicating adenoviral vector. Adenoviruses that do not naturally transduce mesothelial cells and hepatocytes, such as some non-human adenoviruses, can be used in the context of the invention. However, adenoviral vectors based on serotypes of human adenovirus that naturally infect cells of the mesothelium and liver are modified to reduce binding to these cells. By “reduced” transduction or binding is meant that transduction levels of a target cell, such as a mesothelial cell or hepatocyte, by the replication-deficient or conditionally-replicating adenoviral vector is at least approximately 3-fold less (e.g., at least approximately 5-fold, 10-fold, 15-fold, 20-fold, 25-fold, 35-fold, 45-fold, or 50-fold less) than transduction levels mediated by wild-type adenovirus of the same serotype of the replication-deficient or conditionally-replicating adenoviral vector. Preferably, the reduction in transduction efficiency is a substantial reduction (such as at least an order of magnitude, and preferably more). Desirably, the replication-deficient or conditionally-replicating adenoviral vector does not transduce mesothelial cells or hepatocytes.

To reduce native binding and transduction of the replication-deficient or conditionally-replicating adenoviral vector, the native binding sites located on adenoviral coat proteins which mediate cell entry, e.g., the fiber and/or penton base, are absent or disrupted. Two or more of the adenoviral coat proteins are believed to mediate attachment to cell surfaces (e.g., the fiber and penton base). Any suitable technique for altering native binding to a host cell (e.g., a mesothelial cell or hepatocyte) can be employed. For example, exploiting differing fiber lengths to ablate native binding to cells can be accomplished via the addition of a binding sequence to the penton base or fiber knob. This addition can be done either directly or indirectly via a bispecific or multispecific binding sequence. Alternatively, the adenoviral fiber protein can be modified to reduce the number of amino acids in the fiber shaft, thereby creating a “short-shafted” fiber (as described in, for example, U.S. Pat. No. 5,962,311). The fiber proteins of some adenoviral serotypes are naturally shorter than others, and these fiber proteins can be used in place of the native fiber protein to reduce native binding of the adenovirus to its native receptor. For example, the native fiber protein of an adenoviral vector derived from serotype 5 adenovirus can be switched with the fiber protein from adenovirus serotypes 40 or 41.

In another embodiment, the nucleic acid residues associated with native substrate binding can be mutated (see, e.g., International Patent Application WO 00/15823; Einfeld et al., J. Virol., 75(23), 11284-11291 (2001); and van Beusechem et al., J. Virol., 76(6), 2753-2762 (2002)) such that the adenoviral vector incorporating the mutated nucleic acid residues is less able to bind its native substrate. For example, adenovirus serotypes 2 and 5 transduce cells via binding of the adenoviral fiber protein to the coxsackievirus and adenovirus receptor (CAR) and binding of penton proteins to integrins located on the cell surface. Accordingly, the replication-deficient or conditionally-replicating adenoviral vector of the inventive method can lack native binding to CAR and/or exhibit reduced native binding to integrins. To reduce native binding of the replication-deficient or conditionally-replicating adenoviral vector to host cells, the native CAR and/or integrin binding sites (e.g., the RGD sequence located in the adenoviral penton base) are removed or disrupted.

The replication-deficient or conditionally-replicating adenoviral vector also can comprise a chimeric coat protein comprising a non-native amino acid sequence that binds a substrate (i.e., a ligand). As the inventive method allows an adenoviral vector to remain in circulation for extended periods of time, the inventive method is particularly suited for use of “targeted” adenoviral vectors, which comprise a non-native amino acid sequence that preferentially binds a target cell. The non-native amino acid sequence of the chimeric adenoviral coat protein allows an adenoviral vector comprising the chimeric coat protein to bind and, desirably, infect host cells not naturally infected by the corresponding adenovirus without the non-native amino acid sequence (i.e., host cells not infected by the corresponding wild-type adenovirus), to bind to host cells naturally infected by the corresponding adenovirus with greater affinity than the corresponding adenovirus without the non-native amino acid sequence, or to bind to particular target cells with greater affinity than non-target cells. A “non-native” amino acid sequence can comprise an amino acid sequence not naturally present in the adenoviral coat protein or an amino acid sequence found in the adenoviral coat but located in a non-native position within the capsid. By “preferentially binds” is meant that the non-native amino acid sequence binds a receptor, such as, for instance, αvβ3 integrin, with at least about 3-fold greater affinity (e.g., at least about 5-fold, 10-fold, 15-fold, 20-fold, 25-fold, 35-fold, 45-fold, or 50-fold greater affinity) than the non-native ligand binds a different receptor, such as, for instance, αvβ1 integrin.

The non-native amino acid sequence can be conjugated to any of the adenoviral coat proteins to form a chimeric coat protein. Therefore, for example, the non-native amino acid sequence of the invention can be conjugated to, inserted into, or attached to a fiber protein, a penton base protein, a hexon protein, proteins IX, VI, or IIIa, etc. The sequences of such proteins, and methods for employing them in recombinant proteins, are well known in the art (see, e.g., U.S. Pat. Nos. 5,543,328; 5,559,099; 5,712,136; 5,731,190; 5,756,086; 5,770,442; 5,846,782; 5,962,311; 5,965,541; 5,846,782; 6,057,155; 6,127,525; 6,153,435; 6,329,190; 6,455,314; 6,465,253; and 6,576,456; U.S. Patent Application Publication 2001/0047081 and 2003/0099619; and International Patent Applications WO 96/07734, WO 96/26281, WO 97/20051, WO 98/07877, WO 98/07865, WO 98/40509, WO 98/54346, WO 00/15823, WO 01/58940, and WO 01/92549). The coat protein portion of the chimeric coat protein can be a full-length adenoviral coat protein to which the ligand domain is appended, or it can be truncated, e.g., internally or at the C— and/or N-terminus. The coat protein portion need not, itself, be native to the adenoviral vector. For example, the coat protein can be an adenoviral serotype 4 (Ad4) fiber protein incorporated into an adenoviral serotype 5 vector, wherein the native CAR binding motif of the Ad4 fiber is preferably ablated. Likewise, a simian adenovirus type 25 (SAV-25) fiber protein can be incorporated into an adenoviral serotype 35 capsid. Native binding of the SAV-25 fiber can be ablated by mutating the AB loop and β sheet of the fiber protein, and, optionally, a non-native amino acid sequence can be inserted into the H1 loop or attached to the C-terminus of the fiber protein. However modified (including the presence of the non-native amino acid), the chimeric coat protein preferably is able to incorporate into an adenoviral capsid as its native counterpart coat protein. Once a given non-native amino acid sequence is identified, it can be incorporated into any location of the virus capable of interacting with a substrate (i.e., the viral surface). For example, the ligand can be incorporated into the fiber, the penton base, the hexon, protein IX, VI, or IIIa, or other suitable location. Where the ligand is attached to the fiber protein, preferably it does not disturb the interaction between viral proteins or fiber monomers. Thus, the non-native amino acid sequence preferably is not itself an oligomerization domain, as such can adversely interact with the trimerization domain of the adenovirus fiber. Preferably the ligand is added to the virion protein, and is incorporated in such a manner as to be readily exposed to the substrate (e.g., at the N— or C-terminus of the protein, attached to a residue facing the substrate, positioned on a peptide spacer to contact the substrate, etc.) to maximally present the non-native amino acid sequence to the substrate. Ideally, the non-native amino acid sequence is incorporated into an adenoviral fiber protein at the C-terminus of the fiber protein (and attached via a spacer) or incorporated into an exposed loop (e.g., the HI loop) of the fiber to create a chimeric coat protein. Where the non-native amino acid sequence is attached to or replaces a portion of the penton base, preferably it is within the hypervariable regions to ensure that it contacts the substrate. Where the non-native amino acid sequence is attached to the hexon, preferably it is within a hypervariable region (Miksza et al., J. Virol., 70(3), 1836-44 (1996)). Use of a spacer sequence to extend the non-native amino acid sequence away from the surface of the adenoviral particle can be advantageous in that the non-native amino acid sequence can be more available for binding to a receptor and any steric interactions between the non-native amino acid sequence and the adenoviral fiber monomers is reduced.

Binding affinity of a non-native amino acid sequence to a cellular receptor can be determined by any suitable assay, a variety of which assays are known, and is useful in selecting a non-native amino acid sequence for incorporating into an adenoviral coat protein. Desirably, the transduction levels of host cells are utilized in determining relative binding efficiency. Thus, for example, host cells displaying αvβ3 integrin on the cell surface (e.g., MDAMB435 cells) can be exposed to a replication-deficient or conditionally-replicating adenoviral vector comprising the chimeric coat protein and the corresponding adenovirus without the non-native amino acid sequence, and then transduction efficiencies can be compared to determine relative binding affinity. Similarly, both host cells displaying αvβ3 integrin on the cell surface (e.g., MDAMB435 cells) and host cells displaying predominantly αvβ1 on the cell surface (e.g., 293 cells) can be exposed to the adenoviral vectors comprising the chimeric coat protein, and then transduction efficiencies can be compared to determine binding affinity.

The non-native amino acid sequence can bind a particular cellular receptor present on a narrow class of cell types (e.g., tumor cells, cardiac muscle, skeletal muscle, smooth muscle, etc.) or a broader group encompassing several cell types. Through integration of an appropriate cell-specific ligand, the virion can be employed to target any desired cell type, such as, for example, neuronal cells, glial cells, endothelial cells (e.g., via tissue factor receptor, FLT-1, CD31, CD36, CD34, CD105, CD13, ICAM-1 (McCormick et al., J. Biol. Chem., 273, 26323-29 (1998)), thrombomodulin receptor (Lupus et al., Suppl., 2, S 120 (1998)), VEGFR-3 (Lymboussaki et al.,Am. J. Pathol., 153(2), 395-403 (1998), mannose receptor, VCAM-1 (Schwarzacher et al., Atherocsclerosis, 122, 59-67 (1996)), or other receptors), blood clots (e.g., through fibrinogen or aIIbb3 peptide), epithelial cells (e.g., inflamed tissue through selecting, VCAM-1, ICAM-1, etc.), keratinocytes, follicular cells, adipocytes, fibroblasts, hematopoietic or other stem cells, myoblasts, myofibers, cardiomyocytes, smooth muscle, somatic, osteoclasts, osteoblasts, tooth blasts, chondrocytes, melanocytes, hematopoietic cells, etc., as well as cancer cells derived from any of the above cell types (e.g., prostate (such as via PSMA receptor (see, e.g., Schuur et al., J. Biol. Chem., 271 (12), 7043-7051 (1996); Cancer Res., 58, 4055 (1998))), breast, lung, brain (e.g., glioblastoma), leukemia/lymphoma, liver, sarcoma, bone, colon, testicular, ovarian, bladder, throat, stomach, pancreas, rectum, skin (e.g., melanoma), kidney, etc.).

In other embodiments (e.g., to facilitate purification or propagation within a specific engineered cell type), a non-native amino acid (e.g., ligand) can bind a compound other than a cell-surface protein. Thus, the ligand can bind blood- and/or lymph-borne proteins (e.g., albumin), synthetic peptide sequences such as polyamino acids (e.g., polylysine, polyhistidine, etc.), artificial peptide sequences (e.g., FLAG), and RGD peptide fragments (Pasqualini et al., J. Cell. Biol., 130, 1189 (1995)). A ligand can even bind non-peptide substrates, such as plastic (e.g., Adey et al., Gene, 156, 27 (1995)), biotin (Saggio et al., Biochem. J., 293, 613 (1993)), a DNA sequence (Cheng et al.Gene, 171, 1 (1996); Krook et al., Biochem. Biophys., Res. Commun., 204, 849 (1994)), streptavidin (Geibel et al., Biochemistry, 34, 15430 (1995); Katz, Biochemistry, 34, 15421 (1995)), nitrostreptavidin (Balass et al., Anal. Biochem., 243, 264 (1996)), heparin (Wickham et al., Nature Biotechnol., 14, 1570-73 (1996)), or other potential substrates.

Examples of suitable non-native amino acid sequences and their substrates for use in the method of the invention include, but are not limited to, short (e.g., 6 amino acids or less) linear stretches of amino acids recognized by integrins, as well as polyamino acid sequences such as polylysine, polyarginine, etc. Inserting multiple lysines and/or arginines provides for recognition of heparin and DNA. Suitable non-native amino acid sequences for generating chimeric adenoviral coat proteins are further described in U.S. Pat. No. 6,455,314 and International Patent Application WO 01/92549.

Preferably, the adenoviral coat protein comprises a non-native amino acid sequence that binds αvβ3, αvβ5, or αvβ6 integrins. To increase targeting efficiency, native binding of the adenoviral coat protein to native adenoviral cell-surface receptors, such as the coxsackie and adenovirus receptor (CAR), is ablated, as described herein. Preferably, when the non-native amino acid sequence binds (αvβ3 integrin, it does so with at least about 10-fold greater affinity than the non-native amino acid sequence binds to αvβ1 integrin. αvβ3 integrins are upregulated in tumor tissue vasculature, metastatic breast cancer, melanoma, and gliomas. Adenoviral vectors displaying ligands specific for αvβ3 integrin, such as an RGD motif, infect cells with a greater number of αvβ3 integrin moieties on the cell surface compared to cells that do not express the integrin to such a degree, thereby targeting the vectors to specific cells of interest. In one embodiment, the RGD motif is flanked by one or two sets of cysteine residues. In fact, it has been observed that incorporation of an RGD motif (see, e.g., Koivunen et al., Biotechnology, 13, 265 (1995)) into the fiber protein of a replication-deficient adenoviral vector increases transduction of tumor cells with low CAR expression, reduces gene transfer to non-target organs following intraperitoneal administration, and, when the adenoviral vector encodes TNF-α, displays potent anti-tumor activity in a peritoneal cancer model.

Alternatively or in addition, the replication-deficient or conditionally-replicating adenoviral vector comprises a chimeric coat protein comprising a non-native amino acid sequence that binds αvβ6 integrins. αvβ6 integrins are nearly or completely absent on normal epithelium and endothelium, and are upregulated in several carcinomas including lung, colon, and ovarian cancers. Incorporation of an αvβ6 integrin binding motif, RTDLXXL (SEQ ID NO: 1), wherein X can be any amino acid, into an adenoviral fiber protein increases the specificity of the resulting adenoviral vector to cancer cells displaying αvβ6 integrin and allows therapeutically significant levels of gene expression in target tumor tissue. Other αvβ6 integrin-binding motifs can be used as the non-native amino acid sequence for incorporation into the adenoviral coat protein including, but not limited to, αvβ6 integrin-binding motifs of foot and mouth virus (FMV; Jackson et al., J. Virol., 74, 4949-4956 (2000)), LAP-1 amino acid sequence (Munger et al., Cell, 96, 319-328 (1999)), and amino acid sequences described in Kraft et al., J. Biol. Chem., 274, 1979-1985 (1999) including RXDL (SEQ ID NO: 2) and RX₁DLX₁X₁X₂ (SEQ ID NO: 3), wherein X₁ can be any amino acid and X₂ is L, I, F, Y, V, or P.

Tumors often comprise a heterogeneous mass of tumor cells, vasculature, and tumor matrix. The interstitial tumor matrix is composed of collagen, glycosaminoglycans (GAGs), and proteoglycans. To target the replication-deficient or conditionally-replicating adenoviral vector to tumor cells, an adenoviral coat protein of the replication-deficient or conditionally-replicating adenoviral vector can comprise a non-native amino acid sequence that preferentially binds the tumor matrix. Suitable non-native amino acid sequences include, for example, collagen-binding motifs such as WREPSFAMLS (SEQ ID NO: 4) and WREPGRMELN (SEQ ID NO: 5) described in Hall et al., Human Gene Therapy, 11, 983-993 (2000), or other tumor matrix-binding motifs identified by display technologies (e.g., retroviral display libraries). Replication-deficient or conditionally-replicating adenoviral vectors targeted to tumor matrix components collect in the vicinity of tumor cells, thereby increasing the likelihood of tumor cell transduction.

In another embodiment, the adenoviral vector comprises a chimeric virus coat protein not selective for a specific type of eukaryotic cell. The chimeric coat protein differs from a wild-type coat protein by an insertion of a nonnative amino acid sequence into or in place of an internal coat protein sequence, or attachment of a non-native amino acid sequence to the N— or C-terminus of the coat protein. For example, a ligand comprising about five to about nine lysine residues (preferably seven lysine residues) is attached to the C-terminus of the adenoviral fiber protein via a non-coding spacer sequence. In this embodiment, the chimeric virus coat protein efficiently binds to a broader range of eukaryotic cells than a wild-type virus coat, such as described in International Patent Application WO 97/20051. In that a tumor does not comprise a homogenous population of cancer cells, such adenoviral vectors can be preferred in some embodiments.

Of course, the ability of an adenoviral vector to recognize a potential host cell can be modulated without genetic manipulation of the coat protein, i.e., through use of a bi-specific molecule. For instance, complexing an adenovirus with a bispecific molecule comprising a penton base-binding domain and a domain that selectively binds a particular cell surface binding site enables the targeting of the adenoviral vector to a particular cell type.

Suitable modifications to an adenoviral vector are described in U.S. Pat. Nos. 5,543,328, 5,559,099, 5,712,136, 5,731,190, 5,756,086, 5,770,442, 5,846,782, 5,871,727, 5,885,808, 5,922,315, 5,962,311, 5,965,541, 6,057,155, 6,127,525, 6,153,435, 6,329,190, 6,455,314, and 6,465,253, U.S. Published Applications 2001/0047081 A1, 2002/0099024 A1, and 2002/0151027 A1, and International Patent Applications WO 96/07734, WO 96/26281, WO 97/20051, WO 98/07865, WO 98/07877, WO 98/40509, WO 98/54346, WO 00/15823, WO 01/58940, and WO 01/92549.

To further enhance persistence of the replication-deficient or conditionally-replicating adenoviral vector in the bloodstream, the adenoviral fiber protein can be modified to render it less able to interact with the innate or acquired host immune system. For example, one or more amino acids of the native fiber protein can be mutated to render the recombinant fiber protein less able to be recognized by neutralizing antibodies than a wild-type fiber (see, e.g., International Patent Application WO 98/40509 (Crystal et al.)). The fiber also can be modified to lack one or more amino acids mediating interaction with the reticulo-endothelial system (RES). For example, the fiber can be mutated to lack one or more glycosylation or phosphorylation sites, the fiber (or virus containing the fiber) can be produced in the presence of inhibitors of glycosylation or phosphorylation, or the adenoviral surface can be mutated to lack putative heparin sulfate proteoglycan binding domains (see, e.g., Dechecchi et al., Virology, 268, 382-390 (2000) and Dechecchi et al., J. Virol., 75, 8772-8780 (2001)).

Alternatively or in addition, the replication-deficient or conditionally-replicating adenoviral vector is associated at its surface with an immunologically inert molecule(s) to “mask” the adenoviral particle from recognition by antibodies and other mammalian defense/clearance mechanisms such as the RES (see, for example, Moghimi and Hunter, Critical Reviews in Therapeutic Drug Carrier Systems, 18(6), 537-550 (2001)). Inert molecules ideally avoid the immune system, neutralizing antibodies, and other blood-borne proteins, scavenger cells, and the reticuloendothelium system. Inert molecules also can aid in resistance to degradative enzymes. Immunologically-inert molecules include, but are not limited to, a poloxamer, a poloxamine, a poly(acryl amide), a poly(2-ethyl-oxazoline), a poly[N-(2-hydroxylpropyl)methylacrylamide], a poly(vinyl alcohol), a poly(vinyl pyrrolidone), a poly(lactide-co-glycolide), a poly(methyl methacrylate), a poly(butyl-2-cyanoacrylate), or a poly(ethylene glycol) (PEG). With respect to PEG, virion proteins can be conjugated to a lipid derivative of PEG comprising a primary amine group, an epoxy group, or a diacyldlycerol group to reduce collectin and/or opsonin affinity or scavenging by Kupffer cells or other cells of the RES (see, e.g., Kilbanov et al., FEBS Lett., 268, 235 (1990), Senior et al., Biochem. Biophys. Acta., 1062, 11 (1991), Allen et al., Biochem. Biophys. Acta., 1066, 29 (1991), and Mori et al., FEBS Lett., 284, 263 (1991)). Conjugation of immunologically inert molecules to the viral surface is known in the art. For example, PEGylation of adenovirus is described in Croyle et al., J. Virol., 75(10), 4792-4801 (2001), and U.S. Pat. No. 6,399,385 (Croyle et al.). Several variations of PEG molecules are commercially available which utilize different amino acids (e.g., lysine or cysteine) for attachment to the viral surface. To facilitate and control conjugation of PEG molecules to the viral surface, adenoviral coat proteins can be modified to contain such attachment sites. Thus, it is appropriate for the replication-deficient or conditionally-replicating adenoviral vector of the inventive method to comprise one or more cysteine and/or lysine residues genetically incorporated into a coat protein. It also can be advantageous to incorporate non-native amino acid sequences into the adenoviral coat in order to target the replication-deficient or conditionally-replicating adenoviral vector to target cells. It is preferred that such non-native amino acid sequences do not contain attachment sites for PEG molecules, which could result in blockage of cell surface binding sites on the non-native amino acid ligand. Accordingly, in one embodiment, the replication-deficient or conditionally-replicating adenoviral vector is PEGylated, and the non-native amino acid sequence does not comprise a cysteine or a lysine onto which a PEG molecule could attach to the non-native amino acid sequence and impede cellular transduction. This construction strategy allows PEGylation of the viral particle while retaining activity.

Exogenous Nucleic Acid

The replication-deficient or conditionally-replicating adenoviral vector comprises at least one exogenous nucleic acid. Any nucleic acid not native to the adenoviral vector is “exogenous.” The exogenous nucleic acid encodes a peptide that exerts a biological effect in a host cell such as, for example, a peptide that is associated with or treats a biological disorder. The exogenous nucleic acid can be obtained from any source, e.g., isolated from nature, synthetically generated, isolated from a genetically engineered organism, and the like.

In one embodiment of the invention, the replication-deficient or conditionally-replicating adenoviral vector comprises a nucleic acid sequence encoding TNF-α. While other members of the TNF family of proteins, such as Fas ligand and CD40 ligand, have utility in treating a number of diseases, TNF-α has been proven to be an effective anti-cancer agent. The effect of TNF-α on cancer is multifactorial including the induction of apoptosis and tumor necrosis. TNF-α induces adhesiveness of vascular endothelium to neutrophils and platelets and decreases thrombomodulin production (Koga et al., Am. J. Physiol., 268, 1104-1113 (1995)). The result is clot formation in the tumor neovasculature and subsequent hemorrhagic necrosis of the tumors. A nucleic acid sequence encoding TNF-α is described in detail in U.S. Pat. No. 4,879,226. An adenoviral vector encoding human TNF is further described in U.S. Pat. No.6,579,522.

The exogenous nucleic acid can encode an angiogenic peptide. An “angiogenic peptide” is a peptide involved in any process leading to the formation of new blood vessels, e.g., basement membrane breakdown, cell proliferation, cell migration, vessel wall maturation, lumen formation, vessel dilatation, production of mediators, branching of vessels, etc. Suitable angiogenic peptides for use in the inventive method include, but are not limited to, an endothelial mitogen, a factor associated with endothelial migration, a factor associated with vessel wall maturation, a factor associated with vessel wall dilatation, a factor associated with extracellular matrix degradation, or a transcription factor. Endothelial mitogens include, for instance, a vascular endothelial growth factor (VEGF, e.g., VEGF₁₂₁, VEGF₁₄₅, VEGF₁₆₅, VEGF₁₈₉, VEGF₂₀₆, VEGF-II, and VEGF-C), fibroblast growth factors (FGF, e.g., aFGF, bFGF, and FGF-4), platelet derived growth factor (PDGF), placental growth factor (PLGF), angiogenin, hepatocyte growth factor (HGF), tumor growth factor-beta (TGF-β), connective tissue growth factor (CTGF), and epidermal growth factor (EGF). Endothelial migration can be induced by, for example, Del-1. Factors associated with vessel wall maturation include, but are not limited to, angiopoietins (Ang, e.g., Ang-1 and Ang-2), tumor necrosis factor-alpha (TNF-α), midkine (MK), COUP-TFII, and heparin-binding neurotrophic factor (HBNF, also known as heparin binding growth factor). Vessel wall dilatators include, for example nitric oxide synthase (e.g., eNOS and iNOS) and monocyte chemoattractant protein-1 (MCP-1). Extracellular matrix degradation is promoted by, for instance, Ang-2, TNF-α, and MK. Suitable transcription factors include, for instance, HIF-1a and PR39. Other angiogenesis-promoting factors include activin binding protein (ABP) and tissue inhibitor of metalloproteinase (TIMP). Clotting factors, such as tissue factor, FVIIa, FXa, thrombin, and activators of PAR1, PAR2, and PAR3 receptors, also are thought to play a role in angiogenesis (see, for example, Carmeliet et al., Science, 293, 1602 (2001)). Additional angiogenic-promoting factors are described in published U.S. Patent Application No. US2003/0027751 A1.

Angiogenesis-promoting factors are variously described in U.S. Pat. No. 5,194,596 (Tischer et al.), U.S. Pat. No. 5,219,739 (Tischer et al.), U.S. Pat. No. 5,240,848 (Keck et al.), U.S. Pat. No. 5,332,671 (Ferrara et al.), U.S. Pat. No. 5,338,840 (Bayne et al.), U.S. Pat. No. 5,532,343 (Bayne et al.), U.S. Pat. No. 5,169,764 (Shooter et al.), U.S. Pat. No. 5,650,490 (Davis et al.), U.S. Pat. No. 5,643,755 (Davis et al.), U.S. Pat. No.5,879,672 (Davis et al.), U.S. Pat. No. 5,851,797 (Valenzuela et al.), U.S. Pat. No. 5,843,775 (Valenzuela et al.), and U.S. Pat. No. 5,821,124 (Valenzuela et al.); International Patent Applications WO 95/24473 (Hu et al.) and WO 98/44953 (Schaper); European Patent Documents 0 476 983 (Bayne et al.), 0 506 477 (Bayne et al.), and 0 550 296 (Sudo et al.); Japanese Patent Documents 1038100, 2117698, 2279698, and 3178996; J. Folkman et al., Nature, 329, 671 (1987); Fernandez et al., Circulation Research, 87, 207-213 (2000), and Moldovan et al., Circulation Research, 87, 378-384 (2000). Preferably, at least one of the nucleic acid sequences encodes a tissue-specific angiogenic factor, most preferably an endothelial-specific angiogenic factor, such as VEGF.

Alternatively, the exogenous nucleic acid can encode an angiogenesis inhibitor that inhibits or reduces neovascularization in the mammal. Angiogenesis inhibitors can, for example, inhibit cell proliferation, cell migration, vessel formation, extracellular matrix degradation, production of mediators, and the like. Angiogenesis inhibitors also can be antagonists for angiogenesis-promoting agents, such that the angiogenesis-promoting factors are neutralized (see, for example, Sato, Proc. Natl. Acad. Sci. USA, 95, 5843-5844 (1998)).

Angiogenesis inhibitors suitable for use in the inventive method include, for instance, anti-angiogenic factors, cytotoxins, apoptotic factors, anti-sense molecules specific for an angiogenic factor, ribozymes, receptors for an angiogenic factor (e.g., soluble VEGF-R1 (flt-1), soluble VEGF-R2 (flk/kdr), soluble VEGF-R3 (flt-4), and VEGF-receptor-chimeric proteins (Aiello, Proc. Natl. Acad Sci., 92, 10457 (1995))), an antibody that binds an angiogenic factor, and an antibody that binds a receptor for an angiogenic factor. Anti-angiogenic factors include, for instance, angiostatin, thrombospondin, protamine, vasculostatin, endostatin, platelet factor 4, heparinase, interferons (e.g., INFα), and the like. One of ordinary skill in the art will appreciate that any anti-angiogenic factor can be modified or truncated and retain anti-angiogenic activity. As such, active fragments of anti-angiogenic agents (i.e., those fragments having biological activity sufficient to inhibit angiogenesis) are suitable for use in the inventive method. Anti-angiogenic agents are further discussed in U.S. Pat. No 5,840,686; International Patent Applications WO 93/24529 and WO 99/04806; Chader, Cell Different., 20, 209-216 (1987); Dawson et al., Science, 285, 245-248 (1999); and Browder et al, J. BioL Chem., 275, 1521-1524 (2000).

Numerous cytotoxins and apoptotic factors are known in the art and include, for example, p53, Fas, Fas ligand, Fas-associating protein with death domain (FADD), caspase-3, caspase-8 (FLICE), FAIM, Gax, SARP-2, caspase-10, Apo2L, IkB, DIkB, receptor-interacting protein (RIP)-associated ICH-1/CED-3 -homologous protein with a death domain (RAIDD), TNF-related apoptosis-inducing ligand (TRAIL), DR4, DR5, a cell death-inducing coding sequence of Bcl-2 which comprises an N-terminal deletion, a cell death-inducing coding sequence of Bcl-x which comprises an N-terminal deletion, Bax, Bak, Bid, Bad, Bik, Bif-2, c-myc, Ras, Raf, PCK kinase, AKT kinase, Akt/PI(3)-kinase, PITSLRE, death-associated protein (DAP) kinase, RIP, JNK/SAPK, Daxx, NIK, MEKK1, ASK1, PKR, and mutants thereof (e.g., dominant negative mutants thereof and dominant positive mutants thereof), and fragments thereof (e.g., active domains thereof), and combinations thereof. Apoptotic, cytotoxic, and cytostatic transcription factors include, for example, E2F transcription factors and synthetic cell cycle-independent forms thereof, an AP1 transcription factor, an AP2 transcription factor, an SP transcription factor (e.g., an SP1 transcription factor), a helix-loop-helix transcription factor, a DP transcription factor (e.g., DP1, DP2, and DP3), and mutants thereof (e.g., dominant negative mutants thereof and dominant positive mutants thereof), and fragments thereof (e.g., active domains thereof), and combinations thereof. Apoptotic, cytotoxic, and cytostatic viral proteins include, for example, an adenoviral E1A product, an adenoviral E4/ORF6/7 product, an adenoviral E4/ORF4 product, a cytomegalovirus (CMV) product (e.g., CMV-thymidine kinase (CMV-TK)), a herpes simplex virus (HSV) product (e.g., HSV-TK), a human papillomavirus (HPV) product (e.g., HPVX), and mutants thereof (e.g., dominant negative mutants thereof and dominant positive mutants thereof), and fragments thereof (e.g., active domains thereof), and combinations thereof. Cytotoxins and apoptotic factors are particularly useful in inhibiting cell proliferation, an important angiogenic process. Suitable cytotoxins and apoptotic agents can be identified using routine techniques, such as, for instance, cell growth assays and the TUNEL assay, respectively.

The exogenous nucleic acid also can encode pigment epithelium-derived factor (PEDF) or a therapeutic fragment thereof. PEDF, also named early population doubling factor-1 (EPC-1), is a secreted protein having homology to a family of serine protease inhibitors named serpins. PEDF is made predominantly by retinal pigment epithelial cells and is detectable in most tissues and cell types of the body. PEDF has both neurotrophic and anti-angiogenic properties and, therefore, is useful in the treatment and study of a broad array of diseases. Neurotrophic factors are thought to be responsible for the maturation of developing neurons and for maintaining adult neurons. It has been postulated that neurotrophic factors can actually reverse degradation of neurons associated with, for example, vision loss. Neurotrophic factors function in both paracrine and autocrine fashions, making them ideal therapeutic agents. In this regard, PEDF has been observed to induce differentiation in retinoblastoma cells and enhance survival of neuronal populations (Chader, Cell Different., 20, 209-216 (1987)). PEDF further has gliastatic activity or has the ability to inhibit glial cell growth. PEDF also has anti-angiogenic activity. Anti-angiogenic derivatives of PEDF include SLED proteins, discussed in International Patent Application WO 99/04806. It also has been postulated that PEDF is involved with cell senescence (Pignolo et al., J. Biol. Chem., 268 (12), 8949-8957 (1998)). PEDF is further characterized in U.S. Pat. Nos. 5,840,686, 6,319,687, and 6,451,763, and International Patent Applications WO 93/24529, 95/33480, and WO 99/04806. Viral vectors comprising an exogenous nucleic acid encoding PEDF are further described in International Patent Application WO 01/58494.

The exogenous nucleic acid alternatively or additionally can encode a cytokine or chemokine. Cytokines are generally biological factors released by cells which regulate cell-cell interactions, cellular communication, and other cellular activity. Cytokines include, for example, interferons, interleukins, and lymphokines. Chemokines attract and promote movement of cells. Cytokines include, for example, Macrophage Colony Stimulating Factor (e.g., GM-CSF), Interferon Alpha (IFN-α), Interferon Beta (IFN-β), Interferon Gamma (IFN-γ), interleukins (IL-1, IL-2, IL-4, IL-5, IL-6, IL-8, IL-10, IL-12, IL-13, IL-15, IL-16, and IL-18), the TNF family of proteins, Intercellular Adhesion Molecule-1 (ICAM-1), Lymphocyte Function-Associated antigen-3 (LFA-3), B7-1, B7-2, FMS-related tyrosine kinase 3 ligand, (Flt3L), vasoactive intestinal peptide (VIP), and CD40 ligand. Chemokines include, for example, B Cell-Attracting chemokine-1 (BCA-1), Fractalkine, Melanoma Growth Stimulatory Activity protein (MGSA), Hemofiltrate CC chemokine 1 (HCC-1), Interleukin 8 (IL8), Interferon-stimulated T-cell alpha chemoattractant (I-TAC), Lymphotactin, Monocyte Chemotactic Protein 1 (MCP-1), Monocyte Chemotactic Protein 3 (MCP-3), Monocyte Chemotactic Protein 4 (MCP-4), Macrophage-Derived Chemokine (MDC), a macrophage inflammatory protein (MIP), Platelet Factor 4 (PF4), RANTES, BRAK, eotaxin, exodus 1-3, and the like. Cytokines and chemokines are generally described in the art, including the Invivogen catalog (2002), San Diego, Calif.

The exogenous nucleic acid can be the native nucleic acid or cDNA encoding the desired peptide, although modifications and variations of a coding nucleic acid sequence are possible and appropriate in the context of the invention. For example, the degeneracy of the genetic code allows for the substitution of nucleotides throughout polypeptide coding regions, as well as in the translational stop signal, without alteration of the encoded polypeptide. Such substitutable sequences can be deduced from the known amino acid sequence of, for example, TNF-α or the nucleic acid sequence encoding TNF-α and can be constructed by conventional synthetic or site-specific mutagenesis procedures. Synthetic DNA methods can be carried out in substantial accordance with the procedures of Itakura et al., Science, 198, 1056-1063 (1977), and Crea et al., Proc. Natl. Acad. Sci. USA, 75, 5765-5769 (1978). Site-specific mutagenesis procedures are described in Maniatis et al., Molecular Cloning: A Laboratory Manual, Cold Spring Harbor, N.Y. (2d ed. 1989). Alternatively, the nucleic acid sequence can encode a peptide with extensions on either the N— or C-terminus of the protein, so long as the peptide retains biological activity, such as TNF-α's tumoricidal activity described in U.S. Pat. Nos. 4,650,674, 5,795,967, and 5,972,347, as well as European Patents 168,214 and 155,549.

In addition, a nucleic acid sequence encoding a homolog of any of the peptides described here, i.e., any peptide that is more than about 70% identical (preferably more than about 80% identical, more preferably more than about 90% identical, and most preferably more than about 95% identical) to the protein at the amino acid level and displays the same level of activity of the desired peptide, can be incorporated into the replication-deficient or conditionally-replicating adenoviral vector. The degree of amino acid identity can be determined using any method known in the art, such as the BLAST sequence database. Furthermore, a homolog of the protein can be any peptide, polypeptide, or portion thereof, which hybridizes to the protein under at least moderate, preferably high, stringency conditions, and retains biological activity. Exemplary moderate stringency conditions include overnight incubation at 37° C. in a solution comprising 20% formamide, 5×SSC (150 mM NaCl, 15 mM trisodium citrate), 50 mM sodium phosphate (pH 7.6), 5× Denhardt's solution, 10% dextran sulfate, and 20 mg/ml denatured sheared salmon sperm DNA, followed by washing the filters in 1×SSC at about 37-50° C., or substantially similar conditions, e.g., the moderately stringent conditions described in Sambrook et al., supra. High stringency conditions are conditions that use, for example, (1) low ionic strength and high temperature for washing, such as 0.015 M sodium chloride/0.0015 M sodium citrate/0.1% sodium dodecyl sulfate (SDS) at 50° C., (2) employ a denaturing agent during hybridization, such as formamide, for example, 50% (v/v) formamide with 0.1% bovine serum albumin (BSA)/0.1% Ficoll/0.1% polyvinylpyrrolidone (PVP)/50 mM sodium phosphate buffer at pH 6.5 with 750 mM sodium chloride, 75 mM sodium citrate at 42° C., or (3) employ 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 mg/ml), 0.1% SDS, and 10% dextran sulfate at 420° C., with washes at (i) 420° C. in 0.2×SSC, (ii) at 55° C. in 50% formamide and (iii) at 55° C. in 0.1×SSC (preferably in combination with EDTA). Additional details and an explanation of stringency of hybridization reactions are provided in, e.g., Ausubel et al., supra.

The nucleic acid sequence can encode a functional portion of a desired peptide, i.e., any portion of the protein that retains the biological activity of the naturally occurring, full-length protein at measurable levels. For example, a functional TNF-α fragment produced by expression of the nucleic acid sequence of the replication-deficient or conditionally-replicating adenoviral vector can be identified using standard molecular biology and cell culture techniques, such as assaying the biological activity of the fragment in human cells transiently transfected with a nucleic acid sequence encoding the protein fragment. The exogenous nucleic acid also can encode a fusion protein comprising, in part, a protein of interest paired with other, preferably functional peptide portions. For example, to increase the effectiveness of TNF-α in exerting its biological effect on tumor cells, the exogenous nucleic acid can encode a fusion protein comprising TNF-α or a biologically-active fragment thereof fused to a ligand for a cellular receptor found in tumor cells, e.g., a ligand that binds αvβ3, αvβ5, αvβ6, or CD13.

The exogenous nucleic acid is desirably present as part of an expression cassette, i.e., a particular nucleotide sequence that possesses functions which facilitate subdloning and recovery of a nucleic acid sequence (e.g., one or more restriction sites) or expression of a nucleic acid sequence (e.g., polyadenylation or splice sites). The exogenous nucleic acid is preferably located in the E1 region (e.g., replaces the E1 region in whole or in part) or the E4 region of the adenoviral genome. For example, the E1 region can be replaced by a promoter-variable expression cassette comprising an exogenous nucleic acid. The expression cassette is preferably inserted in a 3′-5′ orientation, e.g., oriented such that the direction of transcription of the expression cassette is opposite that of the surrounding adjacent adenoviral genome. However, it is also appropriate for the expression cassette to be inserted in a 5′-3′ orientation with respect to the direction of transcription of the surrounding genome. In addition to the expression cassette comprising the exogenous nucleic acid, the replication-deficient or conditionally-replicating adenoviral vector can comprise other expression cassettes containing other exogenous nucleic acids, which cassettes can replace any of the deleted regions of the adenoviral genome. The insertion of an expression cassette into the adenoviral genome (e.g., into the E1 region of the genome) can be facilitated by known methods, for example, by the introduction of a unique restriction site at a given position of the adenoviral genome. As set forth above, preferably all or part of the E3 region of the adenoviral vector also is deleted.

Preferably, the exogenous nucleic acid comprises a transcription-terninating region such as a polyadenylation sequence located 3′ of angiogenic peptide coding sequence (in the direction of transcription of the coding sequence). Any suitable polyadenylation sequence can be used, including a synthetic optimized sequence, as well as the polyadenylation sequence of BGH (Bovine Growth Hormone), polyoma virus, TK (Thymidine Kinase), EBV (Epstein Barr Virus), and the papillomaviruses, including human papillomaviruses and BPV (Bovine Papilloma Virus). A preferred polyadenylation sequence is the SV40 (Human Sarcoma Virus-40) polyadenylation sequence.

Preferably, the exogenous nucleic acid is operably linked to (i.e., under the transcriptional control of) one or more promoter and/or enhancer elements, for example, as part of a promoter-variable expression cassette. Techniques for operably linking sequences together are well known in the art. Any suitable promoter or enhancer sequence can be used in the context of the invention. Suitable viral promoters include, for instance, cytomegalovirus (CMV) promoters, such as the CMV immediate-early promoter (described in, for example, U.S. Pat. Nos. 5,168,062 and 5,385,839), promoters derived from human immunodeficiency virus (HIV), such as the HIV long terminal repeat promoter, Rous sarcoma virus (RSV) promoters, such as the RSV long terminal repeat, mouse mammary tumor virus (MMTV) promoters, HSV promoters, such as the Lap2 promoter or the herpes thymidine kinase promoter (Wagner et al., Proc. Natl. Acad Sci., 78, 144-145 (1981)), promoters derived from SV40 or Epstein Barr virus, an adeno-associated viral promoter, such as the p5 promoter, and the like. Preferably, the promoter is the CMV immediate-early promoter.

Many of the above-described promoters are constitutive promoters. Instead of being a constitutive promoter, the promoter can be an inducible promoter, i.e., a promoter that is up- and/or down-regulated in response to an appropriate signal. For example, an expression control sequence up-regulated by a chemotherapeutic agent is particularly useful in cancer applications (e.g., a chemo-inducible promoter). In addition, an expression control sequence can be up-regulated by a radiant energy source or by a substance that distresses cells. For example, an expression control sequence can be up-regulated by ultrasound, light activated compounds, radiofrequency, chemotherapy, and cyofreezing. A preferred replication-deficient or conditionally-replicating adenoviral vector according to the invention comprises a chemo-inducible or radiation-inducible promoter operably linked to an exogenous nucleic acid encoding TNF-α. The use of a radiation-inducible promoter enables localized control of TNF-α production, for example, by the administration of radiation to a cell or host comprising the adenoviral vector, thereby minimizing systemic toxicity. Any suitable radiation-inducible promoter can be used in the context of the invention. A preferred radiation-inducible promoter for use in the context of the invention is the early growth region-1 (EGR-1) promoter, specifically the CArG domain of the EGR-1 promoter. The region of the EGR-1 promoter likely responsible for radiation-inducibility is located between nucleotides −550 bp and −50 bp. The EGR-I promoter is described in detail in U.S. Pat. No. 5,206,152 and International Patent Application WO 94/06916. Another suitable radiation-inducible promoter is the c-Jun promoter, which is activated by X-radiation. The region of the c-Jun promoter likely responsible for radiation-inducibility is believed to be located between nucleotides −1.1 kb to 740 bp. The c-Jun promoter and the EGR-1 promoter are further described in, for instance, U.S. Pat. No. 5,770,581.

The promoter also can be a tissue- or cell-specific promoter, such as a tumor cell-selective promoter. Tumor cell-selective promoters suitable for the replication-deficient or conditionally-replicating adenoviral vector include, but are not limited to, the E2F promoter and the DF3 (muc-1) promoter. The promoter also can be selective for endothelial cells associated with tumors, such as the flt-1 promoter.

Dosage and Method of Administration

The dose of replication-deficient or conditionally-replicating adenoviral vector is slowly released into the bloodstream of a mammal. The dose of replication-deficient or conditionally-replicating adenoviral vector will depend on a number of factors, including the size of a target tissue, the extent of any side-effects, the particular route of administration, and the like. Desirably, a single dose of replication-deficient or conditionally-replicating adenoviral vector comprises at least about 1×10⁵ particles (which also is referred to as particle units) to at least about 1×10¹³ particles of the adenoviral vector. The dose preferably is at least about 1×10⁶ particles (e.g., about 4×10⁶-4×10¹² particles), more preferably at least about 1×10⁷ particles, more preferably at least about 1×10⁸ particles (e.g., about 4×10⁸-4×10¹¹ particles), and most preferably at least about 1×10⁹ particles to at least about 1×10¹⁰ particles (e.g., about 4×10⁹-4×10¹⁰ particles) of the adenoviral vector. Alternatively, the dose comprises no more than about 1×10¹⁴ particles, preferably no more than about 1×10¹³ particles, even more preferably no more than about 1×10¹² particles, even more preferably no more than about 1×10¹¹ particles, and most preferably no more than about 1×10¹⁰ particles (e.g., no more than about 1×10⁹ particles). In other words, a single dose of replication-deficient or conditionally-replicating adenoviral vector can comprise about 1×10⁶ particle units (pu), 2×10⁶ pu, 4×10⁶ pu, 1×10⁷ pu, 2×10⁷ pu, 4×10⁷ pu, 1×10⁸ pu, 2×10⁸pu, 4×10⁸ pu, 1×10⁹ pu, 2×10⁹ pu, 4×10⁹ pu, 1×10¹⁰ pu, 2×10¹⁰ pu, 4×10¹⁰ pu, 1×10¹¹ pu, 2×10¹¹ pu, 4×10¹¹ pu, 1×10¹² pu, 2×10¹² pu, or 4×10¹² pu of the replication-deficient or conditionally-replicating adenoviral vector.

The volume of carrier, especially pharmaceutically-acceptable carrier, in which the replication-deficient or conditionally-replicating adenoviral vector is diluted will depend on the size of the mammal and the time period over which the dose of replication-deficient or conditionally-replicating adenoviral vector is administered, typically in a pharmaceutical composition. For example, when the volume of carrier is based on the size or mass of the mammal, the dose of replication-deficient or conditionally-replicating adenoviral vector is administered in a pharmaceutical composition comprising about 20 ml or more of physiologically-acceptable carrier per kilogram (kg) of mammal. Preferably, the pharmaceutical composition comprises about 40 ml or more of physiologically acceptable carrier/kg of mammal, more preferably about 60 ml or more of physiologically acceptable carrier/per kg of mammal. Even more preferably, the pharmaceutical composition comprises about 80 ml or more of physiologically acceptable carrier/per kg of mammal, and most preferably comprises about 100 ml or more of physiologically acceptable carrier/kg of mammal. Alternatively, the volume of pharmaceutical composition administered to a mammal can be calculated based on the surface area of a mammal, a technique routinely used in pharmacology. In this respect, the pharmaceutical composition comprises about 75 ml or more (e.g., about 100 ml or more) of physiologically acceptable carrier per square meter of surface area of the mammal. Preferably, the pharmaceutical composition comprises about 150 ml or more (e.g., about 175 ml or more, about 200 ml or more, or about 250 ml or more) of physiologically acceptable carrier/m² of surface area of the mammal. More preferably, the dose of the replication-deficient or conditionally-replicating adenoviral vector is administered in a pharmaceutical composition comprising 275 ml or more (e.g., 300 ml or more) of physiologically-acceptable carrier/m² of surface area of the mammal. It will be appreciated that smaller volumes of carrier may be appropriate in some embodiments as described in, for example, U.S. Patent Application Publication 2003/0086903.

The dose of replication-deficient or conditionally-replicating adenoviral vector is slowly released into the bloodstream of the mammal. By “slowly released” is meant that a single dose of replication-deficient or conditionally-replicating adenoviral vector is released into the bloodstream of the mammal over the course of at least about 15 minutes. The slow release of the dose of replication-deficient or conditionally-replicating adenovirus allows a greater fraction of the dose of adenoviral vector to circulate in the bloodstream of the mammal than previously achieved, thereby increasing the likelihood of the replication-deficient or conditionally-replicating adenoviral vector reaching target tissue(s). In one embodiment, the dose of replication-deficient or conditionally-replicating adenovirus is continually released into the bloodstream over the course of at least about 30 minutes (e.g., at least about 45, 60, 90, 120, or 150 minutes). Preferably, the dose of replication-deficient or conditionally-replicating adenoviral vector is administered to the mammal over the course of at least about 3 hours (e.g., at least about 3.5, 4, 4.5, 5, 5.5, 6, 6.5, 7, 7.5, 8, 8.5, 9, or 9.5 hours). Also preferably, the dose of replication-deficient or conditionally-replicating adenoviral vector is administered to the mammal over the course of at least about 10 hours.

Slow release into the bloodstream of a mammal can be achieved by a variety of routes of administration, such as those known to one of ordinary skill in the art. The dose of replication-deficient or conditionally-replicating adenoviral vector can be released directly into systemic circulation by intravenous or intraarterial administration. While use of a syringe may not be desirable to administer the dose of replication-deficient or conditionally-replicating adenovirus over the course of at least about 15 minutes, other apparatuses can be employed to facilitate slow release. For example, IV drips and delivery catheter devices attached to a reservoir, infusion pumps, and the like are particularly suited for slow release of substances into systemic circulation. Likewise, many sustained-release implants are suitable for delivering the replication-deficient or conditionally-replicating adenoviral vector into the bloodstream. Microparticles for sustained release of substances in the body often are constructed from biodegradable polymers which release calculated amounts of therapeutic as the microparticle degrades. Sustained-release formulations can comprise, for example, gelatin, chondroitin sulfate, a polyphosphoester, such as bis-2-hydroxyethyl-terephthalate (BHET), or a polylactic-glycolic acid. Sustained release devices and formulations are further described in, for example, U.S. Pat. Nos. 5,378,475, 5,629,008, 5,733,567, 6,506,410, and 6,455,526.

Instead of directly releasing the dose of replication-deficient or conditionally-replicating adenoviral vector into the bloodstream, the dose of replication-deficient or conditionally-replicating adenoviral vector can be indirectly administered to the bloodstream by introducing the replication-deficient or conditionally-replicating adenoviral vector to a region of the mammal that drains into the circulatory system such that the dose of replication-deficient or conditionally-replicating adenovirus is released into the bloodstream over the course of at least about 15 minutes. One such means of indirect systemic delivery comprises administering the dose of adenoviral vector into the lymphatic system. The function of the lymphatics is, in part, maintaining fluid equilibrium in the body. The lymphatic system collects fluid from tissues and returns interstitial fluid to the bloodstream at the thoracic duct. Administering a dose of replication-deficient or conditionally-replicating adenoviral vector to the lymphatic system capitalizes on the body's natural, steady release of substances into the bloodstream.

Many methods of introducing the dose of replication-deficient or conditionally-replicating adenoviral vector to the lymphatics, such as those methods known to the ordinarily skilled artisan, are appropriate for use in the inventive method. For example, the peritoneal cavity is a major source of drainage into the lymphatic system. Parenteral or intraperitoneal delivery of the dose of replication-deficient or conditionally-replicating adenoviral vectors is one method of administration to the bloodstream via the lymphatics. The dose of replication-deficient or conditionally-replicating adenoviral vector can be supplied to the peritoneal cavity using any appropriate means, such as injection or instillation.

Prior to administering the dose of replication-deficient or conditionally-replicating adenoviral vector comprising the exogenous nucleic acid, it can be advantageous to administer a “pre-dose” of a substance which saturates natural innate clearance mechanisms of the mammal, such as an adenoviral vector. The pre-dose can comprise any adenovirus or adenoviral vector constructs described herein, and preferably comprises replication-deficient or conditionally-replicating adenoviral vectors having a reduced ability to transduce mesothelial cells or hepatocytes than a wild-type adenoviral vector of the same serotype. While not desiring to be held to any particular theory, it is believed that the administration of a pre-dose of adenoviral vector increases the persistence of a dose of replication-deficient or conditionally-replicating adenoviral vector by interfering or interacting with a mammal's clearance effector cells, thereby permitting a larger fraction of a dose of replication-deficient or conditionally-replicating adenoviral vectors to reach the bloodstream and remain in circulation. Alternatively or in addition, a pre-dose of adenoviral vector can provoke a tolerance in the mammal to the replication-deficient or conditionally-replicating adenoviral vector. The pre-dose of adenoviral vector can comprise any suitable number of adenoviral particles in any suitable volume of physiologically acceptable carrier, such as the doses of adenoviral vectors and volumes of physiologically acceptable carrier described herein. Likewise, the pre-dose of adenoviral vector can be administered to the mammal using any route of administration, such as intravenous, intraarterial, or intraperitoneal delivery, and can occur at any time prior to the administration of the dose of replication-deficient or conditionally-replicating adenoviral vector, desirably such that the administration of the pre-dose increases the circulation time of the dose of replication-deficient or conditionally-replicating adenoviral vector. The pre-dose is preferably administered about 5 minutes to about 60 minutes (e.g., about 10 minutes to about 45 minutes) prior to the administration of the dose of replication-deficient or conditionally-replicating adenoviral vector. For example, the pre-dose can be administered about 15 minutes to about 30 minutes prior to administering the dose of replication-deficient or conditionally-replicating adenoviral vector.

Normalized Average Bloodstream Concentration

The invention provides a method for enhancing the persistence of adenoviral vectors in systemic circulation, thereby increasing the likelihood of the replication-deficient or conditionally-replicating adenovirus contacting a target tissue. The relative exposure of a target to a therapeutic, including gene transfer vectors, can be determined by calculating the average bloodstream concentration of the therapeutic over a period of time. The average bloodstream concentration is calculated using standard means, as described below.

The amount (concentration) of replication-deficient or conditionally-replicating adenoviral vector in the bloodstream of the mammal (represented as “Cv” with units of [adenoviral vector particles/unit volume of blood]), that is measured at various time points (represented as “T”) following administration of the replication-deficient or conditionally-replicating adenoviral vector at t=0, is plotted to generate a dose curve (Cv versus T). The area under the resulting curve (AUC) of Cv versus T (with units of [(adenoviral vector particles/unit volume)(time)]) is a standard pharmacological measure of the relative exposure of a target to the replication-deficient or conditionally-replicating adenoviral vector. For example, administration of the replication-deficient or conditionally-replicating adenoviral vector at time=0 minutes is followed by measurement of adenoviral vector concentrations in the bloodstream at 10 minutes, 30 minutes, 90 minutes, 180 minutes, 360 minutes, and 1440 minutes post-administration. The concentration of replication-deficient or conditionally-replicating adenoviral vector at each time point is used to plot an adenoviral vector concentration (Cv) versus time (T) curve. The AUC then can be calculated from the plotted curve in accordance with the following equation:

$$AUC = {\int }_{t = 0}^{t = T} C v ⅆ t$$

The average bloodstream concentration (Cv(ave)), expressed as replication-deficient or conditionally-replicating adenoviral vector particles per unit volume of blood over a time period from t=0 to t=T (e.g., 24 hr or 1440 min), is calculated by dividing the AUC by T (i.e., Cv(ave)=AUC/T). Cv(ave) then can be normalized by expression as a percentage of the theoretical bloodstream concentration of replication-deficient or conditionally-replicating adenoviral vector (Cv(0)) obtained if the adenoviral vector was never cleared from the circulation. Cv(0) is obtained by dividing the vector dose (D; expressed in adenoviral vector particles) by the blood volume (Vb) of the mammal (i.e., Cv(0)=D/Vb). The normalized average bloodstream concentration of the replication-deficient or conditionally-replicating adenoviral vector (Cv(ave)%), expressed as a percentage of the theoretical bloodstream concentration of a dose of adenoviral vector that is never cleared from the bloodstream (Cv(0)), is then calculated by dividing Cv(ave) by Cv(0), and multiplying by 100% (i.e., Cv(ave)%=[Cv(ave)/Cv(0)]100%). Cv(ave)% is a convenient measure for comparing the relative bloodstream persistence of two different adenoviral vectors administered to a mammal in the same way.

In the inventive method, the normalized average bloodstream concentration of the replication-deficient or conditionally-replicating adenoviral vector in the bloodstream over a time period of about 24 hours post-administration, expressed as a percentage of the theoretical bloodstream concentration of a dose of adenoviral vector that is never cleared from the bloodstream, is at least about 1% (e.g., at least about 2%). Preferably, the normalized average bloodstream concentration of the replication-deficient or conditionally-replicating adenoviral vector in the bloodstream over a time period of about 24 hours post-administration is at least about 3% (e.g., at least about 4%), more preferably at least about 5% (e.g., at least about 6% or at least about 7%). Even more preferably, the normalized average bloodstream concentration of the replication-deficient or conditionally-replicating adenoviral vector in the bloodstream over a time period of about 24 hours post-administration is at least about 8% (e.g., at least about 9%), and most preferably at least about 10% (e.g., about 11 % or greater).

Alternatively, the normalized average bloodstream concentration for a dose of replication-deficient or conditionally-replicating adenoviral vector can be compared the normalized average bloodstream concentration for an equivalent dose of wild-type adenovirus, an equivalent dose of adenoviral vector of the same serotype as the replication-deficient or conditionally-replicating adenoviral vector but comprising an unmodified viral surface, or an equivalent dose of adenoviral vector having the ability of wild-type adenovirus to infect mesothelial cells or hepatocytes. For instance, the normalized average bloodstream concentration of the replication-deficient or conditionally-replicating adenoviral vector over a time period of about 24 hours post-administration is preferably at least about 5-fold greater (e.g., at least about 6-fold, 7-fold, 8-fold, or 9-fold greater) than the normalized average bloodstream concentration of an equivalent dose of a wild-type adenoviral vector. More preferably, the normalized average bloodstream concentration of the replication-deficient or conditionally-replicating adenoviral vector over a time period of about 24 hours post-administration is preferably at least about 10-fold greater (e.g., at least about 15-fold, 20-fold, 25-fold, 30-fold, 35-fold, 40-fold, or 45-fold greater) than the normalized average bloodstream concentration for an equivalent dose of a wild-type adenoviral vector. Even more preferably, the normalized average bloodstream concentration of the replication-deficient or conditionally-replicating adenoviral vector over a time period of about 24 hours post-administration is preferably at least about 50-fold greater (e.g., at least about 60-fold, 70-fold, 80-fold, 90-fold, or 100-fold greater) than the normalized average bloodstream concentration of an equivalent dose of a wild-type adenoviral vector.

Cancer Therapy

The invention further provides a method of destroying tumor cells in a mammal. The method comprises slowly delivering a dose of a replication-deficient or conditionally-replicating adenoviral vector to the bloodstream of the mammal. The replication-deficient or conditionally-replicating adenoviral vector comprises (a) a nucleic acid sequence encoding a tumoricidal agent and (b) an adenoviral fiber protein which does not mediate adenoviral entry via a coxsackievirus and adenovirus receptor (CAR), as described herein. Tumor cells and/or cells associated with or in close proximity to a tumor are transduced and the tumoricidal agent is produced, thereby destroying tumor cells in the mammal. Many tumoricidal agents are described herein and identified in the art. A preferred tumoricidal agent is TNF-α. Ideally, the target tissue is a solid tumor or a tumor associated with soft tissue (i.e., soft tissue sarcoma), in a human. The tumor can be associated with cancers of (i.e., located in) the oral cavity and pharynx, the digestive system, the respiratory system, bones and joints (e.g., bony metastases), soft tissue, the skin (e.g., melanoma), breast, the genital system, the urinary system, the eye and orbit, the brain and nervous system (e.g., glioma), or the endocrine system (e.g., thyroid or adrenal gland) and is not necessarily the primary tumor. Tissues associated with the oral cavity include, but are not limited to, the tongue and tissues of the mouth. Cancer can arise in tissues of the digestive system including, for example, the esophagus, stomach, small intestine, colon, rectum, anus, liver (e.g., hepatobiliary cancer), gall bladder, and pancreas. Cancers of the respiratory system can affect the larynx, lung, and bronchus and include, for example, non-small cell lung carcinoma. Tumors can arise in the uterine cervix, uterine corpus, ovary vulva, vagina, prostate, testis, and penis, which make up the male and female genital systems, and the urinary bladder, kidney, renal pelvis, and ureter, which comprise the urinary system. The target tissue also can be associated with lymphoma (e.g., Hodgkin's disease and Non-Hodgkin's lymphoma), multiple myeloma, or leukemia (e.g., acute lymphocytic leukemia, chronic lymphocytic leukemia, acute myeloid leukemia, chronic myeloid leukemia, and the like). The recombinant gene transfer vector and methods described herein are, in one embodiment, used in the treatment of ovarian cancer, such that one or more tumors of the ovary are reduced in size or destroyed.

The tumor can be at any stage, and can be subject to other therapies. The replication-deficient or conditionally-replicating adenovirus vectors of the inventive method are useful in treating tumors (i.e., destruction of tumor cells or reduction in tumor size) that have been proven to be resistant to other forms of cancer therapy, such as radiation-resistant tumors. The tumor also can be of any size. The replication-deficient or conditionally-replicating adenoviral vectors of the inventive method mediate reduction of the size of initially large tumors (e.g., 42 cm² (cross-sectional surface area) or 4400 cm³ in volume). Ideally, the inventive method results in cancerous (tumor) cell death and/or reduction in tumor size. It will be appreciated that tumor cell death can occur without a substantial decrease in tumor size due to, for instance, the presence of supporting cells, vascularization, fibrous matrices, etc. Accordingly, while reduction in tumor size is preferred, it is not required in the treatment of cancer.

One advantage of the inventive method over previous cancer therapies is the ability to target tumor cells while better avoiding non-target tissues. Reducing native binding of the replication-deficient or conditionally-replicating adenoviral vector reduces transduction of non-target tissues such as liver, spleen, kidney, and lung, thereby providing a greater fraction of the dose of replication-deficient or conditionally-replicating adenoviral vector available for target tissue, e.g., tumor, transduction. To further enhance efficiency of delivery of a tumoricidal agent to tumor cells, the replication-deficient or conditionally-replicating adenoviral vector can comprise a non-native amino acid sequence (i.e., ligand) incorporated into an adenoviral coat protein, such as an adenoviral fiber protein, which is specific for a cellular receptor expressed in tumor cells. Examples of suitably non-native amino acid sequences include, but are not limited to, non-native amino acid sequences which bind (αvβ3, (αvβ5, and αvβ6 integrins. By practicing the inventive method, a ratio of the level of tumor transduction by the replication-deficient or conditionally-replicating adenoviral vector compared to the level of, for example, liver transduction by the replication-deficient or conditionally-replicating adenoviral vector of at least about 0.1:1 can be achieved. Preferably, the ratio of the level of tumor transduction by the replication-deficient or conditionally-replicating adenoviral vector compared to the level of liver transduction by the replication-deficient or conditionally-replicating adenoviral vector is at least about 0.5:1, most preferably at least about 1:1.

Pharmaceutical Composition

The replication-deficient or conditionally-replicating adenoviral vector is desirably present in a pharmaceutical composition comprising a pharmaceutically acceptable carrier (e.g., a physiologically acceptable carrier). Any suitable pharmaceutically acceptable carrier can be used within the context of the invention, and such carriers are well known in the art. The choice of carrier will be determined, in part, by the particular site to which the pharmaceutical composition is to be administered and the particular method used to administer the pharmaceutical composition.

Suitable formulations include aqueous and non-aqueous solutions, isotonic sterile solutions, which can contain anti-oxidants, buffers, bacteriostats, and solutes that render the formulation isotonic with the blood or other bodily fluid of the intended recipient, and aqueous and non-aqueous sterile suspensions that can include suspending agents, solubilizers, thickening agents, stabilizers, and preservatives. Preferably, the pharmaceutically acceptable carrier is a liquid that contains a buffer and a salt. The formulations can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets. Preferably, the pharmaceutically acceptable carrier is a buffered saline solution.

More preferably, the pharmaceutical composition is formulated to protect the adenoviral vector from damage prior to administration. The particular formulation desirably decreases the light sensitivity and/or temperature sensitivity of the adenoviral vector. Indeed, the pharmaceutical composition will be maintained for various periods of time and, therefore should be formulated to ensure stability and maximal activity at the time of administration. Typically, the pharmaceutical composition is maintained at a temperature above 0° C., preferably at 4° C. or higher (e.g., 4-10° C.). In some embodiments, it is desirable to maintain the pharmaceutical composition at a temperature of 10° C. or higher (e.g., 10-20° C.), 20° C. or higher (e.g., 20-25° C.), or even 30° C. or higher (e.g., 30-40° C.). The pharmaceutical composition can be maintained at the aforementioned temperature(s) for at least 1 day (e.g., 7 days (1 week) or more), though typically the time period will be longer, such as at least 3, 4, 5, or 6 weeks, or even longer, such as at least 10, 11, or 12 weeks, prior to administration to a patient. During that time period, the adenoviral gene transfer vector optimally loses no, or substantially no, activity, although some loss of activity is acceptable, especially with relatively higher storage temperatures and/or relatively longer storage times. Preferably, the activity of the adenoviral vector composition decreases about 20% or less, preferably about 10% or less, and more preferably about 5% or less, after any of the aforementioned time periods.

To this end, the pharmaceutical composition preferably comprises a pharmaceutically acceptable liquid carrier, such as, for example, those described above, and a stabilizing agent selected from the group consisting of polysorbate 80, L-arginine, polyvinylpyrrolidone, α-D-glucopyranosyl α-D-glucopyranoside dihydrate (commonly known as trehalose), and combinations thereof. More preferably, the stabilizing agent is trehalose, or trehalose in combination with polysorbate 80. The stabilizing agent can be present in any suitable concentration in the pharmaceutical composition. When the stabilizing agent is trehalose, the trehalose desirably is present in a concentration of about 2-10% (wt./vol.), preferably about 4-6% (wt./vol.) of the pharmaceutical composition. When trehalose and polysorbate 80 are present in the pharmaceutical composition, the trehalose preferably is present in a concentration of about 4-6% (wt./vol.), more preferably about 5% (wt./vol.), while the polysorbate 80 desirably is present in a concentration of about 0.001-0.01% (wt./vol.), more preferably about 0.0025% (wt./vol.). When a stabilizing agent, e.g., trehalose, is included in the pharmaceutical composition, the pharmaceutically acceptable liquid carrier preferably contains a saccharide other than trehalose. Suitable formulations of the pharmaceutical composition are further described in U.S. Pat. Nos. 6,225,289 and 6,514,943 and International Patent Application WO 00/34444.

In addition, the pharmaceutical composition can comprise additional therapeutic or biologically active agents. For example, therapeutic factors useful in the treatment of a particular indication can be present. Factors that control inflammation, such as ibuprofen or steroids, can be part of the pharmaceutical composition to reduce swelling and inflammation associated with in vivo administration of the adenoviral vector and physiological distress. Immune system suppressors can be administered with the pharmaceutical composition to reduce any immune response to the adenoviral vector itself or associated with a disorder. Alternatively, immune enhancers can be included in the pharmaceutical composition to upregulate the body's natural defenses against disease.

Radiation Therapy

A typical course of treatment for most types of cancer is radiation therapy. Accordingly, the method of the invention can further comprise administering a dose of radiation to a subject. Radiation therapy uses a beam of high-energy particles or waves, such as X-rays and gamma rays, to eradicate cancer cells by inducing mutations in cellular DNA. In that cancer cells divide more rapidly than normal cells, tumor tissue is more susceptible to radiation than normal tissue. Radiation also has been shown to enhance exogenous DNA expression in exposed cells. When the nucleic acid sequence encoding TNF-α is operably linked to a radiation-inducible promoter, radiation potentiates TNF-α production and maintains therapeutic levels of TNF-α at the tumor site continuously throughout the period of radiation therapy, in addition to the additive or synergistic effect of radiation and TNF-α observed in eradicating tumor cells (see, for example, Hersh et al., Gene Therapy, 2, 124-131 (1995), and Kawashita et al., Human Gene Therapy, 10, 1509-1519 (1999)).

Any type of radiation can be administered to a mammal, so long as the dose of radiation is tolerated by the mammal without significant negative side-effects. Suitable types of radiotherapy include, for example, ionizing (electromagnetic) radiotherapy (e.g., X-rays or gamma rays) or particle beam radiation therapy (e.g., high linear energy radiation). Ionizing radiation is defined as radiation comprising particles or photons that have sufficient energy to produce ionization, i.e., gain or loss of electrons (as described in, for example, U.S. Pat. No. 5,770,581). The effects of radiation can be at least partially controlled by the clinician. The dose of radiation is preferably fractionated for maximal target cell exposure and reduced toxicity. Radiation can be administered concurrently with radiosensitizers that enhance the killing of tumor cells, or with radioprotectors (e.g., IL-1 or IL-6) that protect healthy tissue from the harmful effects of radiation. Similarly, the application of heat, i.e., hyperthermia, or chemotherapy can sensitize tissue to radiation.

The source of radiation can be external or internal to the mammal. External radiation therapy is most common and involves directing a beam of high-energy radiation to a tumor site through the skin using, for instance, a linear accelerator. While the beam of radiation is localized to the tumor site, it is nearly impossible to avoid exposure of normal, healthy tissue. However, external radiation is usually well tolerated by patients. Internal radiation therapy involves implanting a radiation-emitting source, such as beads, wires, pellets, capsules, and the like, inside the body at or near the tumor site. Such implants can be removed following treatment, or left in the body inactive. Types of internal radiation therapy include, but are not limited to, brachytherapy, interstitial irradiation, and intracavity irradiation. A less common form of internal radiation therapy is radioimmunotherapy wherein tumor-specific antibodies bound to radioactive material is administered to a patient. The antibodies seek out and bind tumor antigens, thereby effectively administering a dose of radiation to the relevant tissue.

No matter the method of administration, the total dose of radiation administered to a mammal in the context of the invention preferably is about 5 Gray (Gy) to about 70 Gy. More preferably, about 10 Gy to about 65 Gy (e.g., about 15 Gy, 20 Gy, 25 Gy, 30 Gy, 35 Gy, 40 Gy, 45 Gy, 50 Gy, 55 Gy, or 60 Gy) are administered over the course of treatment. While a complete dose of radiation can be administered over the course of one day, the total dose is ideally fractionated and administered over several days. Desirably, radiotherapy is administered over the course of at least about 3 days, e.g., at least 5, 7, 10, 14, 17, 21, 25, 28, 32, 35, 38, 42, 46, 52, or 56 days (about 1-8 weeks). Accordingly, a daily dose of radiation will comprise approximately 1-5 Gy (e.g., about 1 Gy, 1.5 Gy, 1.8 Gy, 2 Gy, 2.5 Gy, 2.8 Gy, 3 Gy, 3.2 Gy, 3.5 Gy, 3.8 Gy, 4 Gy, 4.2 Gy, or 4.5 Gy), preferably 1-2 Gy (e.g., 1.5-2 Gy). The daily dose of radiation should be sufficient to induce expression of the nucleic acid sequence if operably linked to a radiation-inducible promoter. If stretched over a period of time, radiation preferably is not administered every day, thereby allowing the subject to rest and the effects of the therapy to be realized. For example, radiation desirably is administered on 5 consecutive days, and not administered on 2 days, for each week of treatment, thereby allowing 2 days of rest per week. However, radiation can be administered 1 day/week, 2 days/week, 3 days/week, 4 days/week, 5 days/week, 6 days/week, or all 7 days/week, depending on the response of the patient to therapy and any potential side effects.

Chemotherapy

Like radiation, chemotherapy is a standard treatment for reducing the size of a tumor or destroying a tumor. A dose of one or more chemotherapeutics can be administered to a mammal in conjunction with administering a replication-deficient adenoviral vector comprising a nucleic acid sequence encoding TNF-α. A chemotherapeutic agent can be administered before administration of the replication-deficient adenoviral vector, after administration of the replication-deficient adenoviral vector, or concurrently with the replication-deficient adenoviral vector in the same pharmaceutical composition or as a separate administration. Any suitable chemotherapeutic can be used. Suitable chemotherapeutics include, but are not limited to, adriamycin, asparaginase, bleomycin, busulphan, cisplatin, carboplatin, carmustine, capecitabine, chlorambucil, cytarabine, cyclophosphamide, camptothecin, dacarbazine, dactinomycin, daunorubicin, dexrazoxane, docetaxel, doxorubicin, etoposide, floxuridine, fludarabine, fluorouracil, gemcitabine, hydroxyurea, idarubicin, ifosfamide, irinotecan, lomustine, mechlorethamine, mercaptopurine, meplhalan, methotrexate, mitomycin, mitotane, mitoxantrone, nitrosurea, paclitaxel, pamidronate, pentostatin, plicamycin, procarbazine, rituximab, streptozocin, teniposide, thioguanine, thiotepa, vinblastine, vincristine, vinorelbine, taxol, transplatinum, anti-vascular endothelial growth factor compounds (“anti-VEGFs”), anti-epidermal growth factor receptor compounds (“anti-EGFRs”), 5-fluorouracil, and the like. The type and number of chemotherapeutics administered to a subject will depend on the standard chemotherapeutic regimen for a particular tumor type. In other words, while a particular cancer may be routinely treated with a single chemotherapeutic agent, another may be routinely treated with a combination of chemotherapeutic agents. Preferably, the chemotherapeutic agent administered to a subject is selected from the group consisting of 5-fluorouracil (5-FU), cisplatin, paclitaxel, gemcitabine, cyclophosphamide, capecitabine, and/or doxorubicin. Any suitable dose of the one or more chemotherapeutics can be administered to a mammal, e.g., a human. Suitable doses of the chemotherapeutics described above are known in the art, and are described in, for example, U.S. Patent Application Publication No. 2003/0082685 A1. In embodiments where a dose of 5-FU is administered to a human patient, the dose preferably comprises about 50 mg per m² of body surface area of the patient per day (i.e., mg/m^{2 /}day) to about 1500 mg/m²/day (e.g., about 100 mg/m^{2/}day, about 500 mg/m²/day, and about 1000 mg/m²/day). More preferably, the dose of 5-FU comprises about 100 mg/m²/day to about 300 mg/m²/day (e.g., 200 mg/m²/day) or about 900 mg/m^{2 /}day to about 1100 mg/m²/day (e.g., about 1000 mg/m²/day). When a dose of cisplatin is administered to a human patient, the dose preferably comprises about 25 mg/m²/day to about 500 mg/m²/day (e.g., about 50 mg/m²/day, about 100 mg/m^{2 /}day, or about 300 mg/m²/day). More preferably, the dose of cisplatin is about 50-100 mg/m²/day, most preferably 75 mg/m²/day. When a dose of capecitabine is administered to the patient, the dose preferably comprises about 500 mg/m²/day to about 1500 mg/m²/day (e.g., about 700 mg/m²/day, about 800 mg/m²/day, or about 900 mg/m²/day). More preferably, the dose of capecitabine comprises about 800 mg/m²/day to about 1000 mg/m²/day (e.g., about 900 mg/m²/day).

As with radiation, if stretched over a period of time, chemotherapy is not administered every day, thereby allowing the subject to rest and the effects of the therapy to be realized. For example, chemotherapy desirably is administered on 5 consecutive days, and not administered on 2 days, for each week of treatment, thereby allowing 2 days of rest per week. However, chemotherapy can be administered 1 day/week, 2 days/week, 3 days/week, 4 days/week, 5 days/week, 6 days/week, or all 7 days/week, depending on the response of the patient to therapy and any potential side effects.

In some embodiments, it may be advantageous to employ a method of administering the one or more chemotherapeutics wherein a dose is continuously administered to a subject over a prolonged period of time. For example, continuous infusion of the subject with the chemotherapeutic may be desirable. In this regard, the duration of the administration of the dose of the one or more chemotherapeutics may be any suitable length of time. Standard infusion rates for the chemotherapeutics described herein are known in the art and can be modified in any suitable manner according to the nature of the disease. For example, when 5-FU is administered, a typical infusion rate is about 96 hours per treatment week (i.e., 5 days per week). Other aspects of cancer chemotherapy and dosing schedules are described in, for example, Bast et al. (eds.), Cancer Medicine, 5^{th} edition, BC. Decker Inc., Hamilton, Ontario (2000).

The following examples further illustrate the invention but, of course, should not be construed as in any way limiting its scope.

### EXAMPLE 1

This example demonstrates that adenoviral vectors administered to a mammal in accordance with the inventive method persist in circulation for prolonged periods of time.

Adenoviral serotype 5 vectors lacking a majority of coding sequences of the E1 region and E3 region of the adenoviral genome were generated. The replication-deficient adenoviral vectors contain the luciferase reporter gene operably linked to the cytomegalovirus (CMV) promoter (AdL). To reduce adenoviral fiber-mediated transduction via CAR, the AB loop of the adenoviral fiber protein was modified to disrupt CAR binding (AdL.F*). To further reduce native adenovirus-cell surface interaction, the integrin-binding domain of the adenoviral penton base protein was disrupted (AdL.F*PB*). AdL, AdL.F*, and AdL.F*PB*, as well as methods of constructing and propagating adenoviral vectors with reduced native tropism, are further described in Einfeld et al., J. Virol., 75, 11284-11291 (2001).

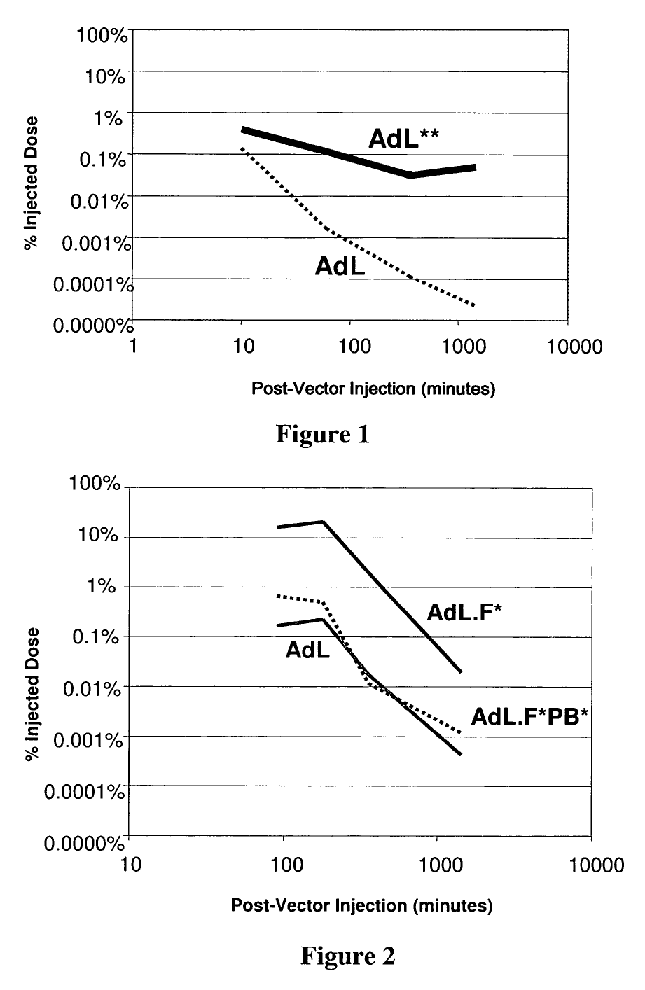

C57B1/6 mice, anesthetized by inhalation of 2-4% isoflurane, were administered a dose of 1×10¹¹ particles of AdL, AdL.F*, or AdL.F*PB* intravenously via the jugular vein. The amount of virus available in the bloodstream was quantitated at 10, 60, 180, and 1440 minutes post-administration. For each time point, the percentage of injected dose was determined and graphed as a function of time post-administration of the vector (see FIG. 1). The area under the resulting curve (AUC) and normalized average bloodstream concentration for each adenoviral vector was calculated as described herein. The resulting data is set forth in Table 1, in which the normalized average bloodstream concentration of AdL and AdL.F*PB* for each time point is represented as “% AUC”.

TABLE 1
IV injection

| | |
|---|---|
| AdL | AdL.F*PB* |

| min. | % AUC | % dose | % AUC | % dose |
|---|---|---|---|---|
| 10 | 3.77 | 0.142 | 6.41 | 0.411 |
| 60 | 0.641 | 0.0017 | 1.25 | 0.116 |
| 180 | 0.214 | 0.0001 | 0.457 | 0.0314 |
| 1440 | 0.0268 | 0 | 0.0916 | 0.0493 |

At 24 hours (i.e., 1440 minutes) post-administration, the normalized average bloodstream concentration (“% AUC”) was less than 1% for both adenoviral vector constructs.

Another population of mice was administered a dose of 1×10¹¹ particles of AdL, AdL.F*, or AdL.F*PB* in 500 μl composition into the peritoneal cavity. The amount of virus present in the bloodstream was quantitated at 90, 180, 360, and 1440 minutes post-administration. For each time point, the percentage of injected dose (“% dose”) was determined and graphed as a function of time post-administration of the vector (see FIG. 2). The normalized average bloodstream concentration of AdL, AdL.F*, and AdL.F*PB* was calculated as described herein and is set forth in Table 2, wherein normalized average bloodstream concentration is represented as “% AUC.”

TABLE 2
IP Injection

| | AdL | | AdL.F* | | AdL.F*PB* | |
|---|---|---|---|---|---|---|
| min. | % AUC | % dose | % AUC | % dose | % AUC | % dose |
| 90 | 0.000 | 0.161 | 0.0001 | 16.2 | 0.000 | 0.662 |
| 180 | 0.0946 | 0.222 | 9.20 | 20.9 | 0.288 | 0.501 |
| 360 | 0.0783 | 0.0173 | 7.79 | 1.95 | 0.182 | 0.0113 |
| 1440 | 0.0216 | 0.0004 | 2.09 | 0.0195 | 0.0481 | 0.0012 |

At 24 hours (i.e., 1440 minutes) post-administration, approximately 0.0004% of the injected dose of AdL was present in circulation. The normalized average bloodstream concentration (“% AUC”) of AdL at 24 hours was approximately 0.022%, i.e., considerably less than 1%. At 24 hours, the normalized average bloodstream concentration of AdL.F* was approximately 2.1 %, and the normalized average bloodstream concentration of AdL.F*PB* was approximately 0.05%. Compared to AdL, the adenoviral coat of which is unmodified, the normalized average bloodstream concentration of AdL.F* at 24 hours was approximately 97-fold that of AdL. The normalized average bloodstream concentration of AdL.F*PB* was approximately 2.2-fold that of AdL.

This example demonstrates intraperitoneal administration of adenoviral vectors modified to reduce native binding to host cell receptors as a route of delivery to systemic circulation reduces the clearance of such vectors from the bloodstream.

### EXAMPLE 2

This example demonstrates that pre-dosing a mammal with adenoviral vector can increase the persistence of a dose of replication-deficient adenoviral vector in circulation.

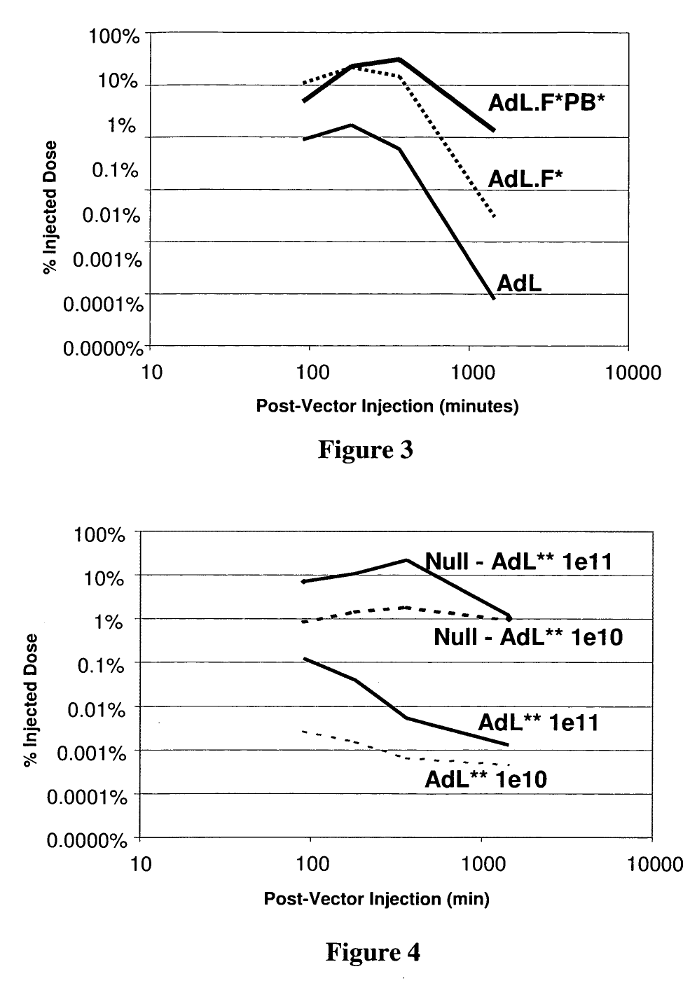

Three populations of mice were anesthetized with 2-4% isoflurane via inhalation and administered a pre-dose of 2×10¹¹ particles of AdNull, an E1/E3-deficient adenoviral lacking a reporter gene and comprising fiber and penton proteins wherein native cell-surface binding sites were disrupted. Ten minutes later (t=0), a dose of 1×10¹¹ particles of one of the three adenoviral vector constructs described in Example 1 was administered in 500 μl of physiologically acceptable carrier. The amount of adenoviral vector in circulation was recorded. For each time point, the percentage of injected dose was determined and graphed as a function of time post-administration of the vector (see FIG. 3). The normalized average bloodstream concentration of AdL, AdL.F*, and AdL.F*PB* was calculated as described herein and is set forth in Table 3, wherein normalized average bloodstream concentration is represented as “% AUC.”

TABLE 3
Pre-dose

| | AdL | AdL.F* | AdL.F*PB* |
|---|---|---|---|
| min. | % AUC | % AUC | % AUC |
| 90 | 0.0000 | 0.0001 | 0.0001 |
| 180 | 0.611 | 7.59 | 5.73 |
| 360 | 0.809 | 12.6 | 17.2 |
| 1440 | 0.219 | 3.65 | 10.1 |

Upon comparison to the data set forth in Table 2, the administration of a pre-dose of adenoviral vector increased the half-life of adenoviral vector in the bloodstream for all three adenoviral vector constructs. The greatest increase in circulation time was observed for AdL.F*PB*, a doubly-ablated adenoviral vector, which enjoyed a 210-fold increase in normalized average bloodstream concentration.

In a separate study, C57B1/6 mice anesthetized under 2-4% isoflurane were intraperitoneally administered a pre-dose of vehicle (10 mM Tris/HCl (pH 7.8) buffer comprising 5% trehalose, 10 mM MgCl₂, and 150 mM NaCl), purified adenoviral hexon protein corresponding to the amount of hexon protein present in a 100 μl composition of 1×10¹¹ adenoviral particles, or 2×10¹¹ particles of AdNull in 100 μl of composition. Ten minutes later (t=0), a dose of 1×10¹⁰ or 1×10¹¹ particles of AdL.F*PB* in 100 ,μl of composition was administered into the peritoneal cavity, as described in Example 1. The amount of AdL.F*PB* in the bloodstream was determined for various time points post-vector administration. For each time point, the percentage of injected dose was determined and graphed as a function of time post-administration of the vector (see FIG. 4). The normalized average bloodstream concentration of AdL.F*PB* was calculated as described herein and is set forth in Table 4, wherein normalized average bloodstream concentration is represented as “% AUC.”

TABLE 4
Pre-dose, AdL.F*PB*

| | Vehicle/Hexon Pre-dose | | AdNull Pre-dose | |
|---|---|---|---|---|
| | (1 × 10¹⁰ pu) | (1 × 10¹¹ pu) | (1 × 10¹⁰ pu) | (1 × 10¹¹ pu) |
| min. | % AUC | % AUC | % AUC | % AUC |
| 90 | 0.00000 | 0.0000 | 0.0000 | 0.0001 |
| 180 | 0.00010 | 0.0349 | 0.432 | 4.50 |
| 360 | 0.00010 | 0.0247 | 0.758 | 10.2 |
| 1440 | 0.00007 | 0.0081 | 0.670 | 6.45 |

Pre-dosing with hexon protein did not have a detectable effect on vector persistence in the bloodstream beyond that observed for pre-dosing with vehicle. Pre-dosing with AdNull increased the normalized average bloodstream concentration for both doses of replication-deficient adenoviral vector administered. At 24 hours post-administration, pre-dosing increased the normalized average bloodstream concentration at least approximately 800-fold compared to the bloodstream concentration of the identical adenoviral vector administered without a pre-dose of adenoviral vector. The results also suggest that an increased dose and volume of composition lead to maximal persistence of adenoviral vector in circulation.

The data provided in this example confirms that administration of a pre-dose of adenoviral vector can further increase the circulation time for a dose of therapeutic adenoviral vector in the bloodstream.

### EXAMPLE 3

This example illustrates a method of modifying an adenoviral vector to further increase half-life in circulation.

The viral surface of AdL.F*PB*, described in Example 1, was coated with PEG molecules. In particular, AdL.F*PB* was desalted by passing the adenoviral vector through a DG column equilibrated with 10 mM potassium phosphate buffer containing 10% sucrose. AdL.F*PB* (9×10¹² particles, 0.25 mg protein) was PEGylated at a ratio of 1:5 and 1:50 (adenoviral protein weight:PEG reagent weight) by addition of 1 mg/ml mPEG-succinimidyl propionate (MW=5000) solution. The PEGylation reaction was terminated by adding excess amount of 10× X lysine. The buffer of PEGylated virus was displaced into 10 mM Tris/HCl (pH 7.8) containing 5% trehalose, 150 mM NaCl, and 10 mM MgCl₂ by passing the vector through a DG column.

A dose of AdL, AdL.F*PB*, AdL.F*PB*(PEG-5), or AdL.F*PB*(PEG-50) (1×10¹¹ pu of adenoviral vector diluted in 500 ,μl of physiologically acceptable carrier) was injected intraperitoneally into mice anesthetized with 2-4% isoflurane. The amount of adenoviral vector in the bloodstream was determined at various time points post-administration. For each time point, the percentage of injected dose was determined and graphed as a function of time post-administration of the vector. The normalized average bloodstream concentration of AdL, AdL.F*PB*, AdL.F*PB*(PEG-5), and AdL.F*PB*(PEG-50) was calculated as described herein and is set forth in Table 5, wherein normalized average bloodstream concentration is represented as “% AUC.”

TABLE 5
PEGylation

| | AdL | AdL.F*PB* | AdL.F*PB*(PEG-5) | AdL.F*PB*(PEG-50) |
|---|---|---|---|---|
| min. | % AUC | % AUC | % AUC | % AUC |
| 60 | 0.0000 | 0.0000 | 0.0000 | 0.0000 |
| 180 | 0.0233 | 0.0973 | 0.0883 | 1.03 |
| 360 | 0.0141 | 0.0781 | 0.0983 | 1.54 |
| 1440 | 0.0038 | 0.0241 | 0.0391 | 0.694 |

PEGylation of the doubly-ablated adenoviral vector increased retention of the adenoviral vector in the bloodstream at least two-fold. The higher concentration of PEG molecules attached to the viral surface further increased the half-life of the adenoviral vector. These results demonstrate that masking the surface of the adenoviral particle reduces clearance of a dose of adenoviral vector when administered in accordance with the inventive method.

### EXAMPLE 4

This example illustrates the ability of the inventive method to efficiently deliver adenoviral vectors comprising a transgene to tumor tissue in vivo.

Nude mice bearing NCI-H441 tumors, a clinically-relevant subcutaneous tumor-bearing animal model, were administered one of four E1/E3-deficient adenoviral vector constructs, all of which comprise the luciferase reporter gene operably linked to the CMV promoter. AdL and AdL.F*PB* are described in Example 1. A ligand which binds (αvβ3 and αvβ5 integrins to mediate viral transduction was inserted into the HI loop of the adenoviral fiber protein of AdL.F*PB* to create AdL**RGD. A ligand which binds αvβ6 (SEQ ID NO: 1) was inserted into the HI loop of the adenoviral fiber protein of AdL.F*PB* to create AdL**αvβ6. The mice were anesthetized via inhalation of 2-4% isoflurane prior to administration of the adenoviral vector.

Two administration strategies were employed to deliver the dose of adenoviral vector. One subset of mice were intravenously administered a dose of 1×10¹¹ particles of adenoviral vector diluted in 100 μl of pharmaceutically acceptable carrier. The remaining mice were injected intraperitoneally with a pre-dose of 2×10¹¹ particles of AdNull, described in Example 2, ten minutes prior to receiving a dose of 1×10¹¹particles of replication-deficient adenoviral vector via intraperitoneal injection. Tumor, liver, spleen, kidney, and/or lung tissue was harvested at 24 hours post-administration of AdL, AdL.F*PB*. AdL**RGD, or AdL**αvβ6. The amount of total protein in the sample was determined by Bio-Rad protein assay and the amount of luciferase activity was determined by luminescence and expressed as relative light units (RLU) per milligram of total protein.

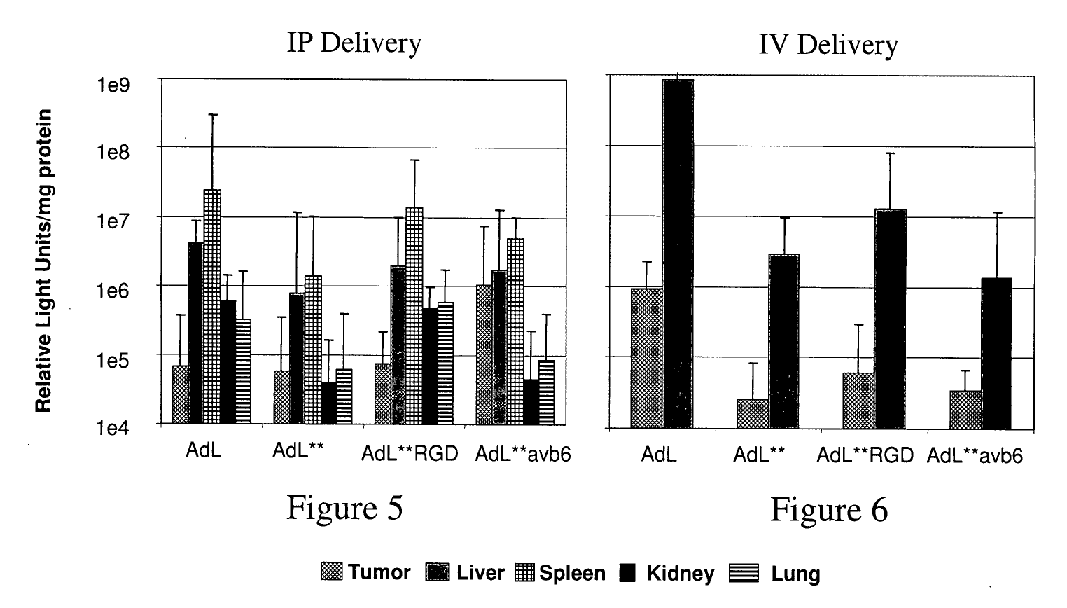

Intensity of luciferase expression was used to quantitate adenoviral vector transduction (see FIGS. 5 and 6). The ratio of tumor transduction to transduction of other tissues was calculated, and is summarized in Table 6.

TABLE 6
Tumor/Tissue Ratio Relative to AdL

| | Intraperitoneal | | | | Intravenous |
|---|---|---|---|---|---|
| | Liver | Spleen | Kidney | Lung | Liver |
| AdL | 0.017 | 0.003 | 0.011 | 0.207 | 0.001 |
| AdL.F*PB* | 0.073 | 0.042 | 1.440 | 0.921 | 0.009 |
| AdL**RGD | 0.038 | 0.005 | 0.156 | 0.130 | 0.005 |
| AdL**αvβ6 | 0.595 | 0.211 | 23.143 | 12.166 | 0.026 |

The ratio of tumor transduction compared to transduction of other tissues was normalized by comparison to the levels of transduction of AdL. The normalized data is set forth in Table 7.

TABLE 7
Tumor/Tissue Ratio Relative to AdL

| | Intraperitoneal | | | | Intravenous |
|---|---|---|---|---|---|
| | Liver | Spleen | Kidney | Lung | Liver |
| AdL | 1 | 1 | 1 | 1 | 1 |
| AdL.F*PB* | 4 | 15 | 13 | 4 | 8 |
| AdL**RGD | 2 | 2 | 1 | 1 | 4 |
| AdL**αvβ6 | 36 | 76 | 213 | 59 | 24 |

This example establishes that the inventive method substantially increases the delivery of gene transfer vector to tumor tissue than intravenous delivery, and provides an alternative to direct injection of gene transfer vector to a tumor. Modifying an adenoviral vector to reduce native binding to cell-surface receptors increases the level of transduction of tumor tissue compared to liver transduction, and insertion of a non-native ligand into the adenoviral fiber protein even further enhances targeting to tumor tissue while avoiding other non-target tissues.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of expressing an exogenous nucleic acid in a mammal, wherein the method comprises slowly releasing into the bloodstream of the mammal a dose of replication-deficient or conditionally-replicating adenoviral vector having a reduced ability to transduce mesothelial cells and hepatocytes compared to wild-type adenovirus and comprising an exogenous nucleic acid,
wherein the normalized average bloodstream concentration of the replication-deficient or conditionally-replicating adenovirus over a time period of 24 hours post-administration, expressed as a percentage of the initial theoretical bloodstream concentration of a dose of adenoviral vector that is never cleared from the bloodstream, is at least about 1%,
such that a host cell in the mammal is transduced and the exogenous nucleic acid is expressed.

2. A method of expressing an exogenous nucleic acid in a mammal, wherein the method comprises slowly delivering to the bloodstream of the mammal a dose of a replication-deficient or conditionally-replicating adenoviral vector having reduced ability to transduce mesothelial cells and hepatocytes compared to wild-type adenoviral vector and comprising an exogenous nucleic acid,
wherein the normalized average bloodstream concentration of the replication-deficient or conditionally-replicating adenoviral vector over a time period of 24 hours post-administration is at least about 5-fold greater than the normalized average bloodstream concentration for an equivalent dose of a wild-type adenoviral vector.

3. The method of claim 1, wherein the replication-deficient or conditionally-replicating adenoviral vector exhibits reduced native binding to a coxsackievirus and adenovirus receptor (CAR).

4. The method of claim 1, wherein the replication-deficient or conditionally-replicating adenoviral vector exhibits reduced native binding to integrins.

5. The method of claim 1, wherein the method comprises releasing the dose of replication-deficient or conditionally-replicating adenoviral vector into the bloodstream over at least about 15 minutes.

6. The method of claim 1, wherein the dose of replication-deficient or conditionally-replicating adenoviral vector is delivered to the bloodstream via the lymphatics or is administered intraperitoneally.

7. The method of claim 6, wherein the method comprises administering a pre-dose of a replication-deficient or conditionally-replicating adenoviral vector prior to administering the dose of replication-deficient or conditionally-replicating adenoviral vector.

8. The method of claim 7, wherein the pre-dose of replication-deficient or conditionally-replicating adenoviral vector is administered intravenously or intraperitoneally.

9. The method of claim 1, wherein the replication-deficient or conditionally-replicating adenoviral vector comprises a chimeric coat protein comprising a non-native amino acid sequence that binds a cellular receptor.

10. The method of claim 9, wherein the chimeric coat protein comprises at least a portion of an adenoviral fiber protein.

11. The method of claim 9, wherein the chimeric coat protein further comprises a spacer.

12. The method of claim 9, wherein the non-native amino acid sequence is incorporated into an exposed loop of the adenoviral fiber protein.

13. The method of claim 9, wherein the non-native amino acid sequence is located at the C-terminus of an adenoviral fiber protein.

14. The method of claim 1, wherein the replication-deficient or conditionally-replicating adenoviral vector is associated at its surface with a poloxamer, a poloxamine, a poly(acryl amide), a poly(2-ethyl-oxazoline), a poly[N-(2-hydroxylpropyl)methylacrylamide], a poly(vinyl alcohol), a poly(vinyl pyrrolidone), a poly(lactide-co-glycolide), a poly(methyl methacrylate), a poly(butyl-2-cyanoacrylate) or a poly(ethylene glycol) (PEG).

15. The method of claim 14, wherein one or more cysteine and/or lysine residues are genetically incorporated into a coat protein of the replication-deficient or conditionally-replicating adenoviral vector.

16. The method of claim 1, wherein the replication-deficient or conditionally-replicating adenoviral vector lacks one or more replication-essential gene functions of the E1 region and the E4 region of the adenoviral genome.

17. The method of claim 1, wherein the host cell is a tumor cell.

18. The method of claim 1, wherein the replication-deficient or conditionally-replicating adenoviral vector comprises a chimeric adenoviral fiber protein comprising a non-native amino acid sequence attached to the C-terminus of an adenoviral fiber protein via a spacer, wherein the non-native amino acid sequence binds a tumor cell receptor on the tumor cell.

19. The method of claim 1, wherein the dose of the replication-deficient or conditionally-replicating adenoviral vector is administered in a pharmaceutical composition comprising 20 ml or more of physiologically acceptable carrier/kg of mammal or 75 ml or more of physiologically acceptable carrier/m² of surface area of the mammal.

20. A method of destroying tumor cells in a mammal, wherein the method comprises slowly delivering a dose of a replication-deficient or conditionally-replicating adenoviral vector to the bloodstream comprising (a) a nucleic acid sequence encoding a tumoricidal agent and (b) an adenoviral fiber protein which does not mediate adenoviral entry via a coxsackievirus and adenovirus receptor (CAR), such that the tumoricidal agent is produced and tumor cells in the mammal are destroyed.

21. The method of claim 20, wherein the replication-deficient or conditionally-replicating adenoviral vector has a reduced ability to transduce mesothelial cells and hepatocytes compared to wild-type adenovirus.

22. The method of claim 20, wherein the dose of replication-deficient or conditionally-replicating adenoviral vector is delivered to the bloodstream via the lymphatics or via administration to the peritoneal cavity.

23. The method of claim 20, wherein the replication-deficient or conditionally-replicating adenoviral vector exhibits reduced native binding to integrins.

24. The method of claim 20, wherein the normalized average bloodstream concentration of the replication-deficient or conditionally-replicating adenoviral vector over a time period of 24 hours post-administration is at least about 3%.

25. The method of claim 20, wherein the replication-deficient or conditionally-replicating adenoviral vector comprises a chimeric coat protein comprising a non-native amino acid sequence that binds a cell surface receptor expressed in a tumor.

26. The method of claim 20, wherein the ratio of the level of tumor transduction by the replication-deficient or conditionally-replicating adenoviral vector compared to the level of liver transduction by the replication-deficient or conditionally-replicating adenoviral vector is at least about 0.1:1.

27. The method any claim 20, wherein the tumoricidal agent is a tumor necrosis factor-alpha.

28. A replication-deficient adenoviral vector comprising a serotype 5 or serotype 35 adenoviral genome with a serotype 41 fiber protein, wherein the replication-deficient adenoviral vector exhibits reduced native binding to integrins.

29. The replication-deficient adenoviral vector of claim 28, wherein the replication-deficient adenoviral vector comprises a penton base protein wherein a native integrin-binding site is mutated.

30. The replication-deficient adenoviral vector of claim 28, wherein the serotype 41 fiber protein exhibits reduced native binding to a coxsackievirus and adenovirus receptor (CAR).

31. The replication-deficient adenoviral vector of claim 30, wherein a native CAR-binding site is mutated.

32. The replication-deficient adenoviral vector of claim 28, wherein the adenoviral vector comprises a serotype 5 adenoviral genome.

33. The replication-deficient adenoviral vector of claim 28, wherein the adenoviral vector comprises a serotype 35 adenoviral genome.
